(19) United States
(12) Patent Application Publication
(10) Pub. No.: US 2005/0072800 A1
(43) Pub. Date: Apr. 7, 2005

(54) Fluid powered proportioning pump and post-mix beverage dispenser system using same

(76) Inventor: Clyde M. Smith, Nashville, TN (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/935,421

(22) Filed: Sep. 8, 2004

Related U.S. Application Data
(60) Provisional application No. 60/503,797, filed on Sep. 19, 2003.
(60) Provisional application No. 60/506,966, filed on Sep. 29, 2003.

(51) Int. Cl. A61M 37/00; B67D 5/56

(52) U.S. Cl. 222/129.2

(57) ABSTRACT

A fluid transfer device includes a housing and a reciprocating piston in the housing. The piston and the housing may be configured to at least partially define at least two variable-volume chambers. An inlet chamber port and an outlet chamber port may be associated with each of the at least two chambers. A valve member may be operatively associated with each inlet chamber port and outlet chamber port to permit or prohibit fluid flow past the respective inlet chamber port and outlet chamber port. The two chambers may be in a state of fluid isolation, such that fluid in one of the chambers does not enter into any other chamber. A valve actuating means may be configured to shift the valve members to permit or prohibit fluid flow past the inlet and outlet chamber ports. A release mechanism may be associated with at least one valve member. The release mechanism may be configured to allow the valve actuating means to shift the valve members to permit or prohibit fluid flow past the inlet or outlet chamber ports. A sub-system in a post-mix beverage dispenser is also disclosed using a fluid powered proportioning pump.

This application claims the benefit of the filing date of U.S. Provisional Application No. 60/503,797, filed Sep. 19, 2003 and U.S. Provisional Application No. 60/506,966, filed Sep. 29, 2003.

### DESCRIPTION OF THE INVENTION

1. Field of the Invention

The present invention relates generally to fluid motor devices capable of converting a portion of the energy of a fluid flow into a reciprocating movement. More particularly, the present invention relates to devices comprised of a fluid motor coupled to a fluid pump. Additionally, the present invention relates to fluid circuits where two or more fluids may be proportioned and may be dispensed.

2. Background of the Invention

Fluid pumps may be powered by electric motors or by fluid motors. Fluid pumps driven by electric motors have a number of undesirable attributes. For example, in some applications, fluid pumps using electric motors may be undesirable for safety reasons. For instance, when pumping solvents, acids, oils, and flammable liquids, it can be disadvantageous or even dangerous to operate high voltage or high current electric motors to drive the pumps. As another example, fluid pumps using electrical motors may not easily start from a stalled condition and stop into a stalled condition. Such intermittent fluid flow is desired in some applications, requiring cyclic starting and stalling of the flow.

Further, existing fluid motors may have valve ports that limit fluid flow through the motor, in a manner that results in pressure drop and turbulence through the motor. When a powering fluid is saturated with a gas, for example, gas saturated carbonated water, too much pressure drop and turbulence through a fluid motor may cause the CO₂ to bubble out of the solution, adversely affecting precision as a fluid powered proportioning pump for carbonated water and syrup. In addition, internal valve leakage in the motor while shifting may adversely affect precision as a fluid powered proportioning pump.

Three devices that may be representative of the art include: a nonelectric proportional dispenser from Dosatron International of 2090 Sunnydale Blvd., 40 Clearwater, Fla. 33765 (phone: 727-443-5404; fax: 727-447-0591); a brix pump from Shurflo of 12650 Westminister Ave., Santa Ana, Calif. 92706-2100 (phone: 714-554-7709; fax: 714-554-4721); and Dosmatic USA of 1230 Crowley Circle, Carrollton, Tex. 75006 (phone: 972-245-9765 fax: 972-245-9000). These devices use water pressure as the power source to operate a fluid powered, piston proportioning pump.

The design of existing post-mix beverage dispensers in the fountain drink systems serving Coca Cola and Pepsi drinks is now fairly standardized around the world and has been so for more than 20 years. Post-mix fountain drinks have a syrup and water component that mix as the drink falls into the serving cup. For drinks using carbonated water as a component, the ratio of syrup to water is approximately 1 to 5. A dispensing valve provides the proportioning function and the on/off function for the water and syrup components. It requires periodic calibration and adjustment.

In existing dispensers, pressurized syrup is provided to a dispensing valve by a gas operated diaphragm pump from an ambient pressure flexible syrup bag in a cardboard box. The powering gas is typically from a pressurized C02 tank. The syrup boxes, tubing, controls and pump are collectively known as a bag-in-box system. The pump is commonly known as a bag-in-box (BIB) pump. The BIB pump may draw from several syrup bags and pump syrup to several dispensing valves.

In existing dispensers, water is provided to a dispensing valve under pressure. If the drink is noncarbonated, the water pressure may be provided by either the existing domestic water line pressure or, if too low, a water pressure booster pump. If the drink is carbonated, the carbonated water pressure results from a carbonator pump and C02 line pressure.

There are commercially available fluid powered proportioning pumps, yet none are suitable for the application described for a number of reasons. The manufacturers include Shurflo, Dosamatic, and Dostron. For the application described, the fluid powered proportioning pump (FPP Pump) must be stable for many years and hours as well as mechanically and volumetrically efficient to be suitable for beverage service. For example, a drift in the volumetric efficiency will result in misproportioned drinks. Mis-proportioned drinks can lead to an unconscious dissatisfaction with the drink and brand long before it is bad enough to attract attention and service. For example, a mechanism that is insufficiently mechanically efficient will result in too great a pressure drop in the carbonated water. As a result, the C02 may bubble out of solution, causing annoying surges of drink into the cup, and may degrade the proportioning accuracy.

In addition, carbonators typically operate at a pressure around or greater than 130 psi. Known FPP pumps may not be rated for this pressure. However, a fluid powered proportioning pump invented by the present inventor is suitable for this application.

### SUMMARY OF THE INVENTION

In one aspect of the invention, a fluid transfer device is disclosed. The fluid transfer device includes a housing and a reciprocating piston in the housing. The piston and the housing may be configured to at least partially define at least two variable-volume chambers. An inlet chamber port and an outlet chamber port may be associated with each of the at least two chambers. A valve member may be operatively associated with each inlet chamber port and outlet chamber port to permit or prohibit fluid flow past the respective inlet chamber port and outlet chamber port. The two chambers may be in a state of fluid isolation, such that fluid in one of the chambers does not enter into any other chamber. A valve actuating means may be configured to shift the valve members to permit or prohibit fluid flow past the inlet and outlet chamber ports. A release mechanism may be associated with at least one valve member. The release mechanism may be configured to allow the valve actuating means to shift the valve members to permit or prohibit fluid flow past the inlet or outlet chamber ports.

The device of the present invention may be used as a fluid motor coupled to a fluid pump. The flow and pressure of a first fluid through a reciprocating piston, fluid motor mechanically coupled to a reciprocating, fluid pump causes a flow and pressure of a second fluid in a predetermined ratio. It is not necessary for the flow and pressure of the first fluid to be the same as the flow and pressure of the second fluid.

The device of the present invention may have utility for a variety of diverse applications without the need of electrical energy. Such applications include, but are not limited to, proportioning, sampling, metering, flow detection, energy recovery, pressure intensification, and pumping. The device of the present invention has advantages of cost, performance, simplicity and materials sufficient to displace devices in existing applications and enable potential new applications. In one example, two or more streams (liquid or gas) will be proportioned where one stream has a source pressure higher than its destination pressure and serves as an energy source to operate a reciprocating motor and the other streams have a source pressure lower than its destination pressure and use a pump powered by a motor to provide pressure and proportioning.

Examples of applications where a fluid pumped or maintained under pressure include the pumping of water, solvents, acids, oils and flammable liquids. As stated above, in some of these applications, an electric motor is disadvantageous for safety reasons. Further, some of these applications require starting from a stalled condition and stopping into a stalled condition and are not easily accomplished with an electric motor. Double diaphragm gas operated pumps may be used. The present invention can use either a pressurized fluid or a pressurized gas.

For applications involving both the pressurization of a fluid and the discharge of a pressurized fluid, waste energy may be reclaimed by the present invention. An example is reverse osmosis where the feed water is pressured by a pump to drive the water through a semi-permeable membrane. Most of the pressurized water is discharged to drain with no re-use of pressurization energy. The present invention may use the energy of the pressurized discharge water to provide most of the pressurization of the feed water of a subsequent membrane by cascading, multiple effect, consequently reducing the pump and motor requirements for feed water pressurization. For example, this is advantageous where available energy is limited such as on submarines or mobile potable, water purification equipment for soldiers.

The system of the present invention may be used in applications where a fluid is de-pressurized and then re-pressured. An example is an ambient pressure solar water heater where ambient temperature, pressurized domestic water is de-pressurized to fill an atmospheric pressure solar collector of the black bag type. Heated water from the collector is re-pressurized to provide flow to faucets and appliances. Use of the present invention, simplifies and lowers the cost of the solar system by using the pressure of the unheated incoming water to re-pressurize the heated collector water in a one to one ratio.

The system of the present invention may be used in applications where a small flow of high pressure fluid is desired and a large flow of low pressure fluid is available. An example is a hydraulic intensifier where the energy of a high flow of low pressure hydraulic oil may be used to operate the fluid motor of the present invention which in turn operates a smaller cross section pump to create a low flow of high pressure hydraulic oil. The increase in pressure is proportional to the ratio of the flows and therefore the cross sections of the motor and pump.

The system of the present invention may be used in dispensing applications, where two or more fluids are mixed in a predetermined adjustable or non-adjustable ratio. Examples include: diluting and mixing herbicides and pesticides into water for agricultural spraying, diluting and mixing fertilizer into irrigation water for agricultural and horticultural use, diluting and mixing soap concentrate into water for washing equipment for clothes, dishes, parts and the like, diluting and mixing an oil concentrate into water for machine tool lubrication, and for the addition of chemical into the make-up water of process tanks. The present invention may function in this application by using the flow and pressure of the water as the first fluid for the fluid motor. The additive fluid as the second fluid is pumped by the fluid pump in a ratio proportional to the cross sections of the fluid motor and fluid pump times their effective stroke. The discharge of both may then be mixed.

The system of the present invention may be used in applications where two or more fluids are mixed in a selectable, adjustable or non-adjustable ratio. An exemplary system includes a fluid motor driving multiple fluid pumps where the pumps are selectably operable. A pump may be made selectably operable by means of closing a pump's discharge with a valve, thereby diverting the flow through a relief valve. A relief valve may be internal or external to its pump. In the case of an internal relief valve, the flow will be diverted upon reaching its relieving pressure from pump outlet to inlet. Alternatively, a pump may be made selectably operable by means of a diverting valve returning a pumped fluid from its downstream side to its upstream side. The means of selectability are exemplary and other means are considered. An exemplary application system is a post mix dispenser using a fluid powered proportioning pump with multiple pumps. Each pump is associated with a separate syrup or supplemental flavor. With selectability enabled at a dispenser valve, a dispenser valve may supply a drink proportioned with water and one syrup or another, as selected. Additionally, a supplemental flavoring may be added to the drink by selection, simultaneously with dispensing of the drink. Supplemental flavors may be added to the drink in selectable amounts by the length of time that the flavor's pump is enabled. A dispenser may include one or more proportioning pumps of the described arrangement.

The system of the present invention may be used in applications that require indicating a flow rate or totalizing a flow. The flow to be measured is used as the fluid through the fluid motor. At least one sensor detects the reciprocating motion of the piston. The signal from the sensor provides information convertible into flow rate or total flow information. A sensor may be provided that can detect the piston motion without direct contact, without a shaft penetrating the housing.

The system of the present invention may be used in sampling applications. The fluid to be sampled powers the fluid motor. The fluid pump will draw some of the discharge fluid of the fluid motor as a second fluid. The flow from the fluid pump will be the sample. The sampling ratio is proportional to the ratio of the cross section of the fluid pump and motor.

A common application particularly suited to the present invention is the post mix beverage dispenser. Since pressurized domestic water is almost always available as a powering fluid, the device of the present invention may be substituted for the compressed, gas operated diaphragm pump by using the domestic water as an energy source and then sending the spent water to drain or re-use. For beer dispensing, an exemplary circuit may include domestic water as the powering fluid for the present invention while beer is the pumped fluid delivered to a dispensing valve. Alternatively, a powering fluid may be contained in a closed circuit that includes a pressure booster pump. For post mix dispensing, an exemplary circuit may include domestic water as the powering fluid for the present invention while syrup is the pumped fluid delivered to the dispensing valve. Using the energy of the carbonated water to pump and proportion the syrup may make an even more advantageous use of the present invention. In this way, the present invention replaces both the conventional syrup pump and the proportioning part of the dispensing valve, further simplifying the dispenser and lowering its manufacturing cost.

The system of the present invention may be used in applications such as fuel cells that use different phases. Compressed air can power the reciprocating motor that powers a fuel pump and a water pump in a desired stoichiometric proportion. The discharge of all three may be then directed into a reformer.

The present invention has advantages in these applications because no electricity is required for power or control. The invention operates as a fluid motor, a fluid pump, a combined fluid motor and pump, or proportioning device as needed by the application. The present invention ceases operation when the flow of one or more fluids is ended, and the invention will resume operation when the flow is re-established, the invention can maintain a pumped fluid pressure, i.e. the device is stallable.

The present invention may have a fluid motor with a tie-rod design, similar in construction to common air and hydraulic tie rod designs and actuating cylinders. This construction is easy and economical to manufacture and capable of withstanding pressures ranging from 100 psi up into the thousands. The present invention may be used in applications under such pressures. The present invention may have a hydraulically balanced multi-port valve with substantially no internal leakage when shifting. This feature provides several important advantages over products currently commercially available: (1) The force required to shift the multi-port valve does not vary substantially with either an increasing pressure differential across the motor or increase of the valve port cross sectional area; (2) therefore, a given valve actuation mechanism and a given valve release mechanism can operate over a wider range of flows and pressures than fluid motors now manufactured; and (3) since there is no design penalty for larger valve port cross sections, a motor of the present invention can be made with large valve ports thereby reducing the pressure drop and turbulence through a motor.

Thus, the present invention aids in avoiding two problems that limit the utility and applications of other designs: (1) when a powering fluid is saturated with a gas, like carbonated water is, too much pressure drop and turbulence through a fluid motor will cause the C02 to bubble out of solution and adversely affect precision as a fluid powered proportioning pump for carbonated water and syrup; and (2) a multi-port valve allowing internal leakage while shifting also adversely affects precision as a fluid powered proportioning pump.

The present invention is easily compatible with embodiments having different features. The present invention describes two exemplary types of valve actuation means: spring and magnetic, although other types of actuation means are considered. The present invention describes two exemplary types of hydraulically balanced valves. The present invention describes three exemplary types of valve release mechanisms: mechanical trigger, mechanical over-the-center, and magnetic, although other types of release mechanisms are considered. The present invention may include a stroke compensator to make the ratio of a fluid powered proportioning pump with double acting pump the same in both stroke directions. The present invention contemplates the use of single acting, double acting, external, internal, fixed ratio, adjustable ratio, multiple, and single pumps. The present invention describes a stroke signal means usable for flow control, flow rate measurement and flow totalizing.

This invention may provide a device functioning as a combined fluid motor and pump of unusual simplicity and low part count.

This invention is consistent with requirements for injection molded, polymer parts and automated or mechanized assembly allowing the potential for low cost mass production. It is also consistent with requirements for metallic parts in applications where stresses are too great for polymer parts.

This invention has numerous useful embodiments suitable for a wide range of applications. Although the invention may be used with additional features, some exemplary embodiments include the following:
(1) fluid motor with a direct or indirect stroke sensor;
(2) fluid motor with shaft and external pump or pumps;
(3) fluid motor without shaft and internal adjustable pump or pumps;
(4) fluid motor with internal and external pumps;
(5) fluid motor with external pump resistant to cross-contamination;
(6) hydraulically balanced multi-port valve; and
(7) low leakage multi-port valve.

In addition, the present invention concerns the design of dispensers. A common example of which is postmix soft drink dispensers where a fluid powered proportioning pump may replace a gas operated diaphragm pump and the proportioning function of a dispenser valve. The present invention may allow a simpler, lower cost, more reliable, lower maintenance, higher performance dispenser design. The present invention enables a dispenser design with no need for electricity. Desirable features such as portion control and flow totalizing may be easily integrated at low cost. The present invention describes controls appropriate to a dispenser using a fluid powered proportioning pump. A dispenser of the present invention substitutes a free, already available power source, carbonated water, in place of compressed air or compressed C02 used by a gas operated diaphragm pump. A dispenser design of the present invention can be made more rugged and simple than existing designs making it more suitable for locations lacking service and support. Yet, it is compatible with features making it more appropriate to sophisticated markets.

It is object of the present invention to provide a device that converts a first fluid flow and pressure into mechanical motion by means of a fluid motor part of the device and using this mechanical motion converts it into fluid flow and pressure by means of a fluid pump part of the device.

It is another object of the present invention to provide a fluid motor where the energy for valve actuation is supplied by piston displacement.

It is another object of the present invention to provide a fluid motor where the energy for valve actuation is stored and released in springs.

It is another object of the present invention to provide a fluid motor where valve actuation is enabled by proximity of the piston to the end of its chamber where contact with a release mechanism allows the shift of a multi-port valve.

It is another object of the present invention to provide a device that operates without need of electrical control or power.

It is another object of the present invention to provide a device that will stop and restart in response to a first fluid's flow and pressure.

It is another object of the present invention to provide a device that will stop and restart in response to a second fluid's flow and pressure.

It is another object of the present invention to provide a fluid motor that will stop and restart in response to input fluid flow and pressure.

It is another object of the present invention to provide a fluid motor that will stop and restart in response to shaft pressure.

It is another object of the present invention to provide accurate, volumetric proportioning of two or more fluids.

It is another object of the present invention to provide fluid motor driven proportioning pump of a fixed ratio.

It is another object of the present invention to provide a fluid motor driven proportioning pump of an adjustable ratio.

It is another object of the present invention to provide a device where the fluids may be either liquid or gas.

It is another object of the present invention to provide a device with a means of sensing the reciprocating motion for control and measurement purposes.

It is another object of the present invention to provide fluid circuits with means of delivering multiple fluids from multiple sources to multiple destinations.

The above objects are exemplary only, and this invention contemplates devices and systems that may meet or fulfill one or more of these objects. Additional objects and advantages of the invention will be set forth in part in the description which follows, and in part will be obvious from the description, or may be learned by practice of the invention. In addition to the structural and procedural arrangements set forth above, the invention could include a number of other arrangements such as those explained hereinafter. It is to be understood that both the foregoing general description and the following detailed description are exemplary only.

### BRIEF DESCRIPTION OF THE DRAWINGS

The accompanying drawings, which are incorporated in and constitute a part of this specification, illustrate embodiments of the invention and together with the description, serve to explain some principles of the invention.

FIG. 1 is a pictorial representation showing a trimetric, general arrangement of an exemplary motor combined with an external, fixed displacement pump in one exemplary embodiment of the present invention.

FIG. 2 is a pictorial representation showing a right view, cross section of an exemplary motor with a piston in an intermediary position to illustrate operation.

FIG. 3 is a pictorial representation showing a right view, cross section of an exemplary motor with a piston in an intermediary position to illustrate operation and multi-port valve assembly that is totally hydraulically balanced.

FIG. 4 is a pictorial representation showing a right view, cross section of an exemplary motor with a piston in an end of stroke position to illustrate operation.

FIG. 5 is a pictorial representation showing a right view, cross section of an exemplary motor with a piston in an intermediary position to illustrate operation.

FIG. 6 is a pictorial representation showing a right view, cross section of an exemplary motor similar to the exemplary motor of FIG. 5, where a mechanical release is replaced by a magnetic release and a stroke sensor is added.

FIG. 7 is a pictorial representation showing a right view, cross section of an exemplary motor similar to the exemplary motor of FIG. 5 combined with an external fixed displacement pump and the mechanical release is of the over-the-center type.

FIG. 8 is a pictorial representation showing a right view, cross section of an exemplary motor similar to that of the exemplary motor of FIG. 6 modified with an internal adjustable displacement pump.

FIG. 9 is a schematic of a post-mix dispenser system with its most basic elements and a single dispenser valve.

FIG. 10 is a schematic of the post-mix dispenser system of FIG. 9 with additional features of multiple dispensing valves and a control mechanism.

FIG. 11 is a schematic of a carbonator using a fluid powered booster pump.

### DESCRIPTION OF THE EMBODIMENTS

Reference will now be made in detail to exemplary embodiments of the invention, an example of which is illustrated in the accompanying drawings. Wherever possible, the same reference numbers will be used throughout the drawings to refer to the same or like parts.

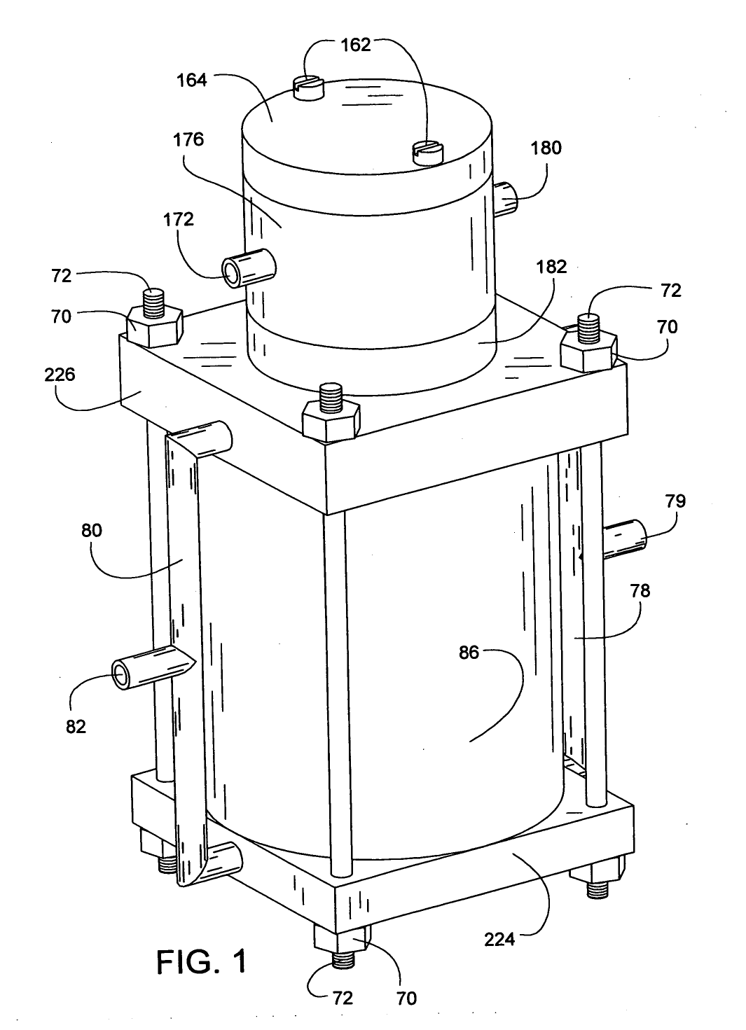

FIG. 1 shows a trimetric, general arrangement of a fluid motor combined with an exemplary external, close coupled, fixed displacement pump in an exemplary embodiment of the present invention. In this exemplary embodiment, an exemplary fluid motor housing may be comprised of an end cap 226, a case 86 and an end cap 224. The housing may be held together in compression with tie rods 72 and nuts 70. Other methods or systems may be used to hold the housing together. A powering fluid may enter through an inlet port 82 and is distributed to end cap 226 and end cap 224 by an inlet manifold 80. The powering fluid may exit from end cap 226 and end cap 224 through outlet manifold 78 and finally outlet port 79.

An exemplary fluid pump is comprised of pump housing segments 164, 176, and 182. Segment 176 may contain pump inlet port 172 and pump outlet port 180. Segments 164, 176, and 182 and end cap 226 may be held together in compression by fasteners 162. Other methods of holding different components would be apparent to one skilled in the art. Pump ports 172 and 180 are shown as tubes suitable for the “instant” type tube connection. These ports along with fluid motor ports 82 and 84 could just as well be another type of fitting, such as, for example, a screw, flange or flare.

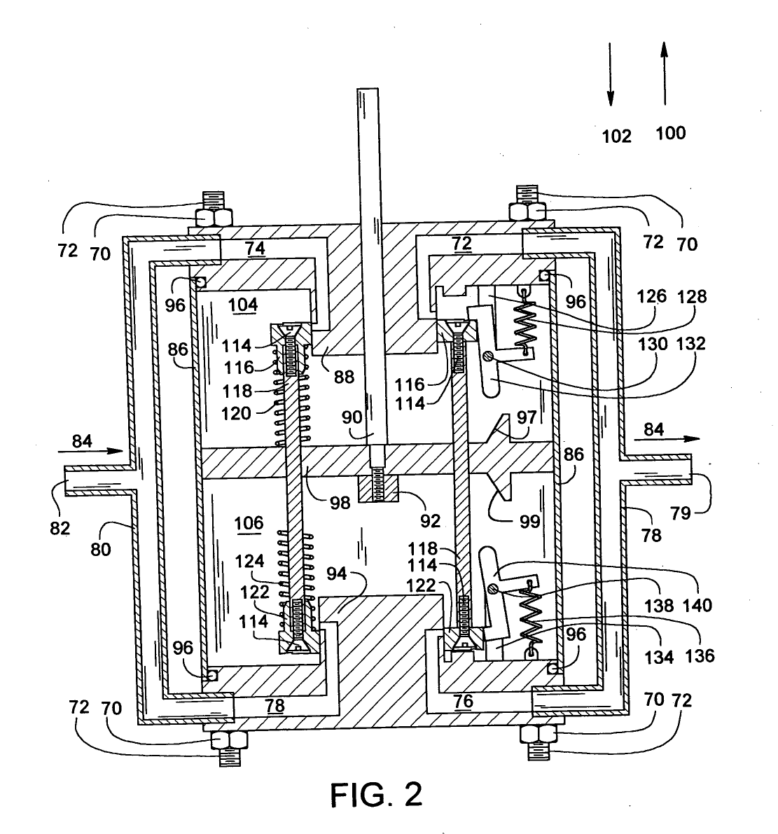

FIG. 2 shows a right view, cross section of an exemplary motor with a piston in an intermediary position to illustrate operation. A housing assembly is comprised of a case 86 with a cylindrical interior surface. One end of the case is closed by end cap 88 and the other end is closed by end cap 94. The interior of the housing is sealed by o-rings seals 96. The housing assembly may be comprised of case 86, end cap 88, end cap 94 and seals 96 and may be held together by any known method, including threaded tie rods 72 and nuts 70. A powering fluid enters inlet port 82 in direction 84. The powering fluid is delivered to end cap 88 and end cap 94 through inlet manifold 80. The powering fluid is collected from end cap 88 and end cap 94 through outlet manifold 78. The powering fluid leaves through outlet port 79 in direction 84.

The interior of a housing assembly is divided into a first variable volume chamber 104 and a second variable volume chamber 106 by a piston assembly. The piston assembly may be comprised of a shaft 90, a piston 98, and a threaded fastener 92 that fixes shaft 90 to piston 98. In this exemplary embodiment, the piston 98 has a first wedge shaped projection 97 on one side and a second wedge shaped projection 99 on the other side for engagement with a valve release assembly. Shaft 90 slidably, sealably pierces end cap 88.

An exemplary multi-port valve assembly is comprised of valve ring 116, valve ring 122, valve connecting rods 118, actuating spring 120, actuating spring 124, and screw fasteners 114. In this exemplary embodiment, valve ring 116 is connected to valve ring 122 by valve connecting rods 118 that slidably, sealably pierce piston 98. Fasteners, such as screw fasteners 114, fix rods 118 to ring 116 and ring 122. A boss on ring 116 may support actuating spring 120. A boss on ring 122 may support actuating spring 124. An alternative exemplary embodiment of a multi-port valve (not shown) is comprised of a ring valve sealing on its outside circumference against case 86 in the same way that piston 98 seals against case 86. A chamber inlet hole or alternatively, a radially disposed set of holes, may pierce case 86 and communicate with inlet manifold 80. A chamber outlet hole or alternately, a radially disposed set of holes, may pierce case 86 and communicate with outlet manifold 84. A chamber inlet hole and a chamber outlet hole may be axially offset from each other. A powering fluid may enter and exit a piston chamber through the case rather than end cap as described previously. This embodiment may be advantageous when miniaturization is a consideration. Other exemplary valve systems would be apparent to one skilled in the art.

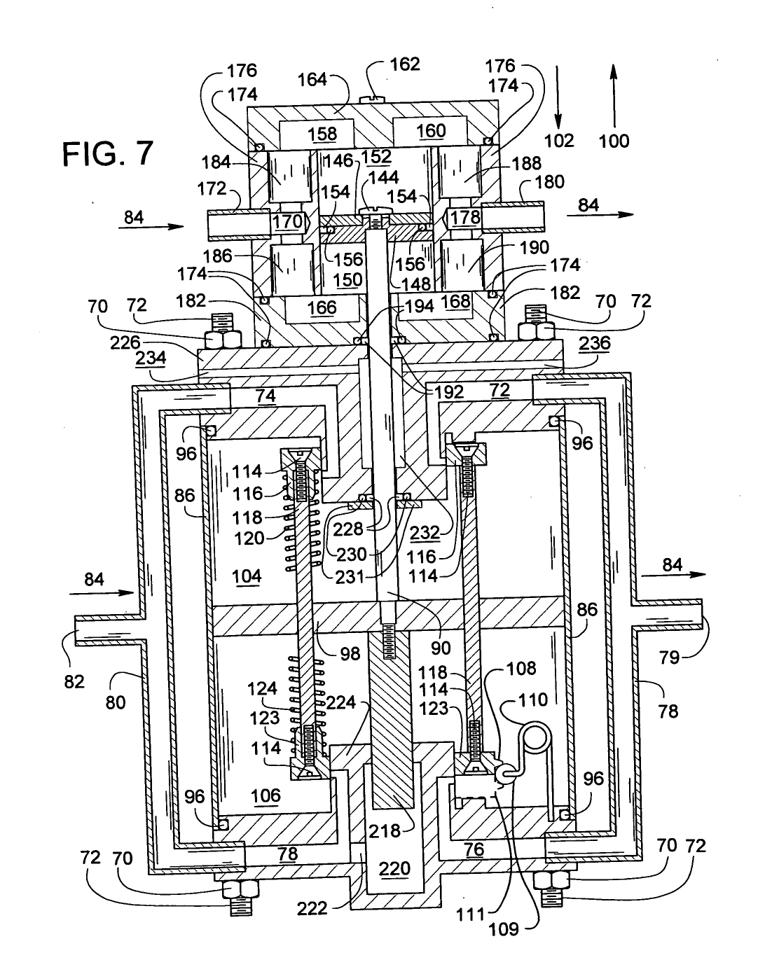

An exemplary release mechanism is comprised of support 126, release shaft 130, catch member 132 and position biasing spring 128. Another exemplary release mechanism is comprised of support 134, release shaft 138, catch member 140 and position biasing spring 136. Catch member 140 may be rotationally biased by spring 136 to capture valve ring 122. Catch member 132 may be rotationally biased by spring 128 to capture valve ring 116. If not blocked by valve ring 116, passageway 74 in end cap 88 may allow a powering fluid to enter chamber 104 from inlet manifold 80. If not blocked by valve ring 122, passageway 78 in end cap 94 may allow a powering fluid to enter chamber 106 from inlet manifold 80. If not blocked by valve ring 116, passageway 72 in end cap 88 may allow a powering fluid to exit chamber 104 to outlet manifold 78. If not blocked by valve ring 122, passageway 76 in end cap 94 may allow a powering fluid to exit chamber 106 to outlet manifold 78. An alternative embodiment of a release mechanism (not shown) is comprised of a single release mechanism that acts to release the valve assembly at the end of each reciprocating stroke of the piston assembly and immediately re-capture the valve assembly upon completion of the valve assembly's shift. This embodiment may be advantageous when miniaturization is a consideration. FIG. 7 shows a single release mechanism located in only one of the two piston chambers acting on the multi-port valve assembly as a double acting release mechanism of the over-the-center type. Other exemplary release mechanisms would be apparent to one skilled in the art.

The multi-port valve assembly may have two resting positions: at its limit of travel in direction 102 and at its limit in direction 100. When the multi-port valve assembly is at its limit of travel in direction 102, it is blocked from additional travel in direction 102 by contact of valve ring 122 with end cap 94. When the multi-port valve assembly is at its limit of travel in direction 102, it is also blocked from additional travel in direction 100 by engagement of valve ring 116 and catch member 132. When the multi-port valve assembly is at its limit of travel in direction 102, passageway 74 is blocked by valve ring 116 from communicating with chamber 104, passageway 76 is blocked by valve ring 122 from communicating with chamber 106, passageway 72 is not blocked by valve ring 116 and communicates freely with chamber 104, and passageway 78 is not blocked by valve ring 122 and freely communicates with chamber 106.

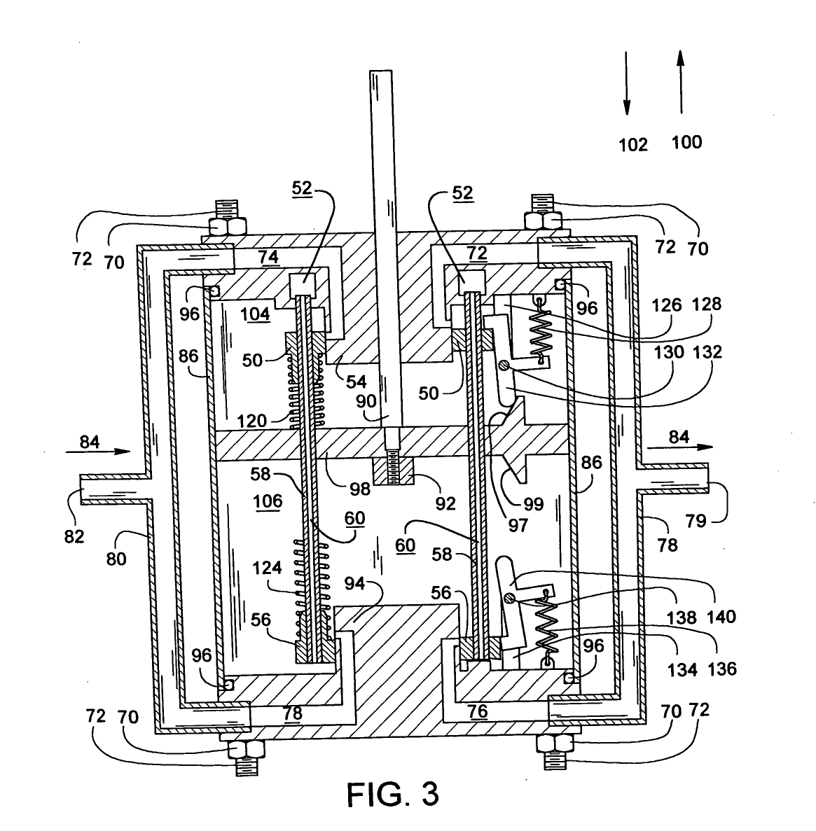

When the multi-port valve assembly is at its limit of travel in direction 100, passageway 72 is blocked by valve ring 116 from communicating with chamber 104, passageway 78 is blocked by valve ring 122 from communicating with chamber 106, passageway 74 is not blocked by valve ring 116 and communicates freely with chamber 104, and passageway 76 is not blocked by valve ring 122 and freely communicates with chamber 106. The valve rings are hydraulically balanced. The force required to shift the valve rings does not significantly change with the change in pressure differential from inlet to outlet. The force required to shift the valve rings does not significantly change with the change in pressure differential from piston chamber to piston chamber. However, valve linkage rods 118 that link the valve rings are not hydraulically balanced. The net force on the linkage does change with the change of pressure differential from piston chamber to piston chamber. The net force effect may be minimized and may be made inconsequential by using valve linkage rods small in cross sectional area. FIG. 3 shows an exemplary valve linkage that is hydraulically balanced.

When the valve assembly shifts from one resting position (limit of travel) to its other resting position (limit of travel), the overlap of valve 116 substantially blocks both passageway 74 and passageway 72. Additionally, the overlap of valve 122 substantially blocks both passageway 78 and passageway 76. Thus, passageway 74 and passageway 72 never substantially communicate with one another through chamber 104, and also passageway 78 and 76 never substantially communicate with one another through chamber 106. This feature increases the efficiency and accuracy of a fluid motor by minimizing internal leakage.

In use, a powering fluid may enter inlet port 82 and continue through inlet manifold 80 to enter passageway 78 as passageway 74 is blocked. From passageway 78, a powering fluid may enter chamber 106, thereby causing piston 98 to move in direction 100 as passageway 76, is blocked. As piston 98 moves in direction 100, chamber 104 decreases in volume and a powering fluid within the chamber 104 may be expelled through passageway 72 as passageway 74 is blocked. The powering fluid may continues from passage 72 into outlet manifold 78 and then exits through outlet port 79 as passageway 76 is blocked. The advance of piston 98 in direction 100 may eventually cause contact with, and then compression of, spring 120. The continued advance of piston 98 in direction 100 further compresses spring 120 so that it is squeezed between piston 98 and valve ring 116 since valve ring 116 is restrained from movement in direction 100 by catch member 132 of the release mechanism.

It should be noted that these exemplary embodiments of the present invention are compatible with a powering gas, such as a compressed gas, as a substitute for a powering liquid. Accordingly, where the word fluid is used, it is meant to include any of gas, liquid, and also a mixed phase.

FIG. 3 shows a right view, cross section of an exemplary motor with a piston in an intermediary position to illustrate operation and modification to make a multi-port valve assembly substantially totally, hydraulically balanced. A powering fluid may enter inlet port 82 and continue through inlet manifold 80 to enter passageway 78 as passageway 74 is blocked. From passageway 78, a powering fluid enters chamber 106 causing piston 98 to move in direction 100 as passageway 76 is blocked. As piston 98 moves in direction 100, chamber 104 decreases in volume and a powering fluid within the chamber 104 is expelled through passageway 72 as passageway 74 is blocked. The powering fluid may continue from passage 72 into outlet manifold 78 and then may exit through outlet port 79 as passageway 76 is blocked.

The advance of piston 98 in direction 100 may eventually cause contact with, and then compression, of spring 120. The continued advance of piston 98 in direction 100 may further compress spring 120 so that it is squeezed between piston 98 and valve ring 50 since valve ring 50 is restrained from movement in direction 100 by catch member 132 of a said release mechanism.

A wedge projection 97 on piston 98 may make contact with a surface of catch member 132 causing the beginning of a clockwise rotation as a result of a wedging action (simple machine incline with relative motion). The wedging action may eventually create a force that overcomes the biasing of spring 128.

In the exemplary embodiment shown, end cap 54 contains a valve linkage rod balancing chamber 52. A valve assembly is comprised of valve rings, valve actuators, and valve linkages, specifically, valve ring 50, valve ring 56, valve spring 120, valve spring 124, and valve linkage rods 58. Valve ring 50 and valve ring 56 may be fixed to valve linkage rods 58. Rods 58 may slidably pierce end cap 54 and may isolate chamber 52 from chamber 104. As shown, rods 58 may have internal passageways 60 that allow communication of chamber 106 with chamber 52. This state may be maintained through all positions of a valve assembly. This arrangement enables the cross sections of valve linkage rods 58 to experience the same pressure of chamber 106. In this way, the valve linkage is hydraulically balanced in addition to the valve rings. An alternative exemplary embodiment of a hydraulically balanced valve linkage (not shown) is comprised of valve linkage rods 118 that extend from valve ring 116 to slidably, sealably pierce end cap 88 and that extend from valve ring 122 to slidably, sealably pierce end cap 94 so that the cross sectional area of rods 118 is acted upon by atmospheric pressure equally in direction 100 and direction 102.

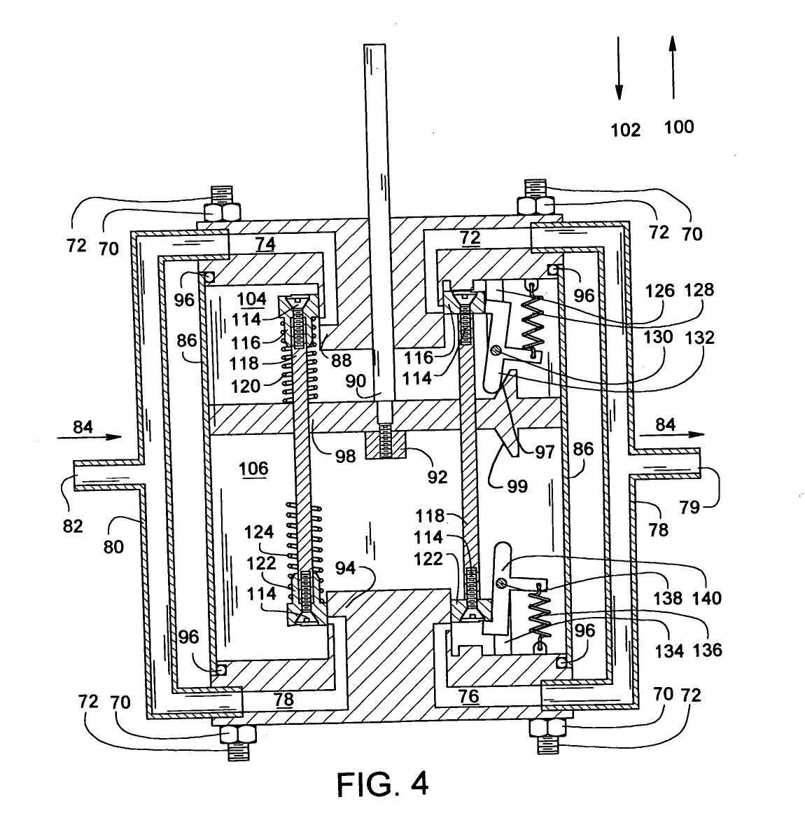

FIG. 4 shows a right view, cross section of an exemplary motor. In FIG. 4, the piston 98 is shown at the end of its stroke to illustrate operation. As shown in FIG. 4, the piston 98 has advanced to cause compression of spring 120 and wedging action of projection 97 on catch member 132. When the piston 98 is sufficiently advanced, the wedging action causes catch member 132 to rotated clear of valve ring 116. With valve ring 116 no longer restrained, the stored energy of compressed spring 120 may act on valve ring 116 to move it to its limit of travel against end cap 88 in direction 100. Valve ring 122 may shift to its limit in direction 100 since it is connected to valve ring 116 by valve connecting rods 118. When valve ring 122 shifts, catch member 140 rotates into it because of the bias provides by spring 136. Thus, the valve assembly is restrained in its alternate limit of travel. Since a powering fluid now enters chamber 104 and exits chamber 106, piston 98 has reversed the direction of its movement. As would be apparent to one skilled in the art, additional methods and systems may be used to restrain the valve assembly and to release it by the movement of the piston. In addition, additional methods and systems for shifting the valve ring would be apparent to one skilled in the art.

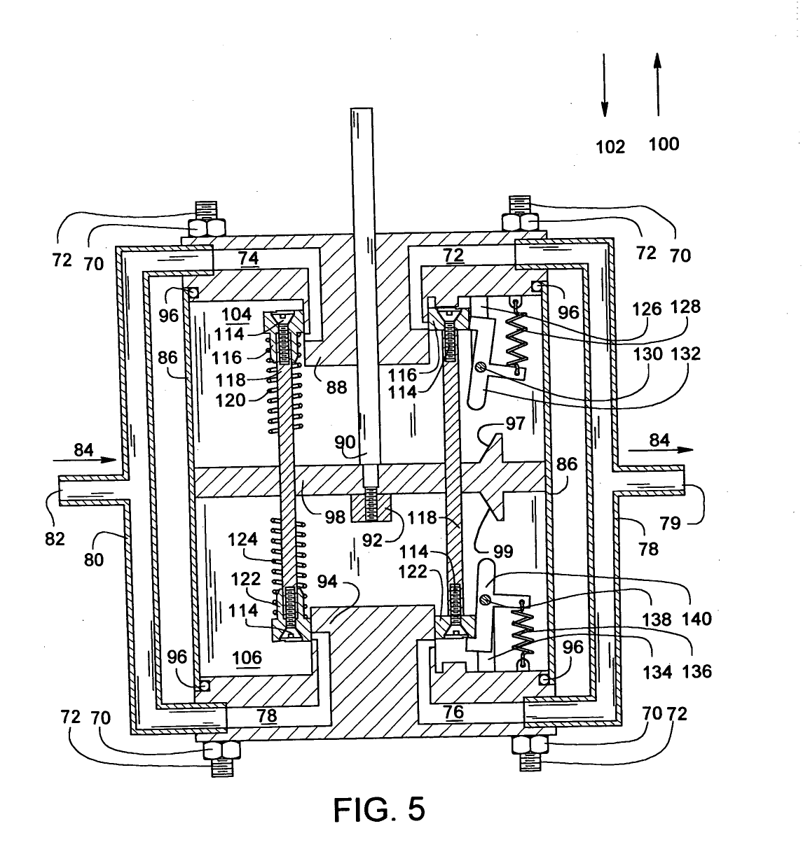

FIG. 5 shows a right view, cross section of an exemplary motor with a piston at an intermediate stroke position to illustrate operation. In this exemplary embodiment, a powering fluid enters inlet port 82 and continues through inlet manifold 80 to enter passageway 74 as passageway 78 is blocked. From passageway 74, a powering fluid enters chamber 104 causing piston 98 to move in direction 102 as passageway 72 is blocked. As piston 98 moves in direction 102, chamber 106 decreases in volume and a powering fluid within is expelled through passageway 76 as passageway 78 is blocked. A powering fluid continues from passage 76 into outlet manifold 78 and then exits through outlet port 79 as passageway 72 is blocked.

As the piston 98 advances in direction 102, it may eventually advance enough to compress actuating spring 124. From there, it may continue its advance until it contacts and triggers a release mechanism that may allow the multi-port valve to shift, thereby reversing the piston stroke. The reciprocating cycle may then repeat.

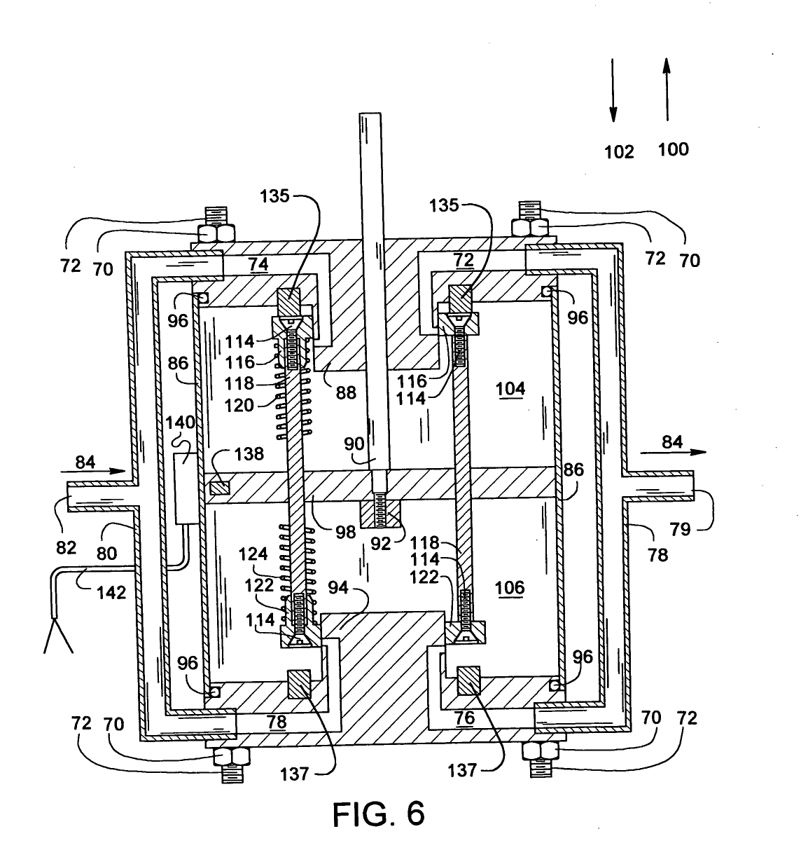

FIG. 6 shows a right view, cross section of an exemplary motor having many features similar to those in the exemplary motor of FIG. 5. Some of the differences include the replacement of a mechanical release with a magnetic release and the addition of a stroke sensor to provide a signal for use in flow measurement and totalizing. The exemplary release mechanism disclosed above with reference to FIG. 5 includes support 126, release shaft 130, catch member 132, and position biasing spring 128. In the exemplary release mechanism of FIG. 6, these are replaced by magnet 135. Likewise, the exemplary release mechanism of FIG. 5 that includes support 134, release shaft 138, catch member 140, and position biasing spring 136 is replaced in FIG. 6 with a magnet 137. This modification anticipates that valve ring 116 and valve ring 122 are comprised of a magnetic material. If not, then an additional magnet may be attached to valve ring 116 and valve ring 122 to establish an attractive magnetic field. Other release mechanisms would be apparent to one skilled in the art.

In the exemplary motor of FIG. 6, a magnet 138 is attached to piston 98 to establish a magnetic field that is detectable outside case 86 by detector 140, such as a reed switch. A signal cable 142 may connect detector 140 to additional signal processing device such as a flow rate meter, flow totalizing meter, or sending unit. Because the stroke, and therefore displacement per stroke, is well defined, counting the strokes provides good correlation for a calculated flow as compared with the actual flow.

In use, a powering fluid enters inlet port 82 and continues through inlet manifold 80 to enter passageway 74 as passageway 78 is blocked. From passageway 74, a powering fluid enters chamber 104 causing piston 98 to move in direction 102 as passageway 72 is blocked. As piston 98 moves in direction 102, chamber 106 decreases in volume and a powering fluid within is expelled through passageway 76 as passageway 78 is blocked. A powering fluid continues from passage 76 into outlet manifold 78 and then exits through outlet port 79 as passageway 72 is blocked.

Piston 98 may advance in direction 102, continuing in its advance and eventually compressing actuating spring 124. While spring 124 undergoes compression, the multi-port valve assembly (comprised of valve ring 116, valve ring 122, rods 118, and fasteners 114) may be held at its limit of travel resting position in direction 100 by the attractive force between magnet 135 and valve ring 116. The attractive force between magnet 137 and valve ring 122 may be comparatively weak because of the distance separating them. As spring 124 is further compressed by the advance of piston 98 in direction 102, the stored energy and force increases.

By design, the force of spring 124 will eventually equal, and then exceed, the countering force of magnetic attraction between magnet 135 and valve ring 116. When this happens, said valve assembly will rapidly shift in direction 102 by the force of spring 124. When said valve assembly reaches the limit of travel in direction 102, the attractive force between magnet 137 and valve ring 122 will hold it there. The shifting of the multi-port valve assembly may re-direct the flow of the powering fluid, thereby causing piston 98 to reverse its stroke direction. The reciprocating cycle may then repeat.

FIG. 7 shows a right view, cross section of the exemplary motor of FIG. 5 with exemplary modifications including: (1) the addition of an external, fixed displacement, double acting, fluid pump; (2) shaft displacement compensator; (3) shaft seal isolation; and (4) a mechanical release of the over-the-center type. An exemplary housing assembly is comprised of a case 86 with a cylindrical interior surface. One end of the case is closed by end cap 226 and the other end is closed by end cap 224. The interior of the housing is sealed by o-rings seals 96. The housing assembly comprised of case 86, end cap 224, end cap 226 and seals 96 may be held together by threaded tie rods 72 and nuts 70. Other systems or methods may be used to hold the separate components of the housing components together.

In use, a powering fluid enters inlet port 82 in direction 84. The powering fluid may be delivered to end cap 224 and end cap 226 through inlet manifold 80. The powering fluid may be collected from end cap 224 and end cap 226 through outlet manifold 78. The powering fluid may leave through outlet port 79 in direction 84.

The interior of the exemplary housing assembly may be divided into a variable volume chamber 104 and another variable volume chamber 106 by a piston assembly. The piston assembly may be comprised of a shaft 90, a piston 98, and a displacement compensator 218 that fixes shaft 90 to piston 98. Shaft 90 may slidably pierce end cap 226. A shaft seal for shaft 90 may be comprised of actuating o-ring 230, rod seal ring 228, and retainer 231. Another shaft seal for shaft 90 may be comprised of actuating o-ring 194 and rod seal ring 192. In the exemplary embodiment shown, a chamber 232 is formed between the shaft seals, further isolating a powering fluid and a pumped fluid. The distance between the shaft seals may be such that a powering fluid and a pumped fluid never share a common shaft surface. A passageway 236 and a passageway 234 may communicate with chamber 232. Passageways 234 and 236 may provide a weephole, air gap or conduit for a barrier fluid in chamber 232. The barrier fluid may be static, flowing or a special purpose fluid like sanitizer. Although the exemplary motor and pump of FIG. 7 is shown in fluid isolation along the shaft seal, such isolation is not a requirement for operability. A simple seal between pump and motor may also be sufficient. The shaft seal isolation is shown as an example only to demonstrate one example incorporating it where it may be desired.

A exemplary multi-port valve assembly may be comprised of valve ring 116, valve ring 123, valve connecting rods 118, actuating spring 120, actuating spring 124, and screw fasteners 114. It may also comprise other components or features. In the exemplary embodiment shown, valve ring 116 is connected to valve ring 123 by valve connecting rods 118 that slidably pierce piston 98. Screw fasteners 114 fix rods 118 to ring 116 and ring 123. A boss on ring 116 supports actuating spring 120. A boss on ring 123 supports actuating spring 124.

An exemplary release mechanism is shown in FIG. 7, and may comprise a roller 109 and a torsion spring 110 fixed to end cap 224 that interacts with an over-the-center projection 108 on valve ring 123. The force exerted on projection 108 by roller 109 and supplied by spring 110, may bias said valve assembly to its travel limit in direction 100. In the exemplary embodiment shown, the bias can be overcome by the increasing force of valve spring 124 as it is compressed by piston 98. When the bias is overcome, the valve assembly will rapidly shift to its alternate travel limit 111. The valve assembly is then biased in direction 102 and is set up for a repetitive cycle. Other release mechanisms may be used as would be apparent to one skilled in the art.

In the exemplary embodiment shown, if not blocked by valve ring 116, passageway 74 in end cap 226 allows a powering fluid to enter chamber 104 from inlet manifold 80. If not blocked by valve ring 123, passageway 78 in end cap 224 allows a powering fluid to enter chamber 106 from inlet manifold 80. If not blocked by valve ring 116, passageway 72 in end cap 226 allows a powering fluid to exit chamber 104 to outlet manifold 78. If not blocked by valve ring 123, passageway 76 in end cap 224 allows a powering fluid to exit chamber 106 to outlet manifold 78.

The exemplary multi-port valve assembly has two resting positions: at its limit of travel in direction 102 and at its limit in direction 100. When the multi-port valve assembly is at its limit of travel in direction 102, it may be blocked from travel in direction 102 by contact of valve ring 123 with end cap 224. When the multi-port valve assembly is at its limit of travel in direction 102, it may also blocked from travel in direction 100 by bias of release mechanism roller 109 bearing on projection 108 when at travel limit 111. When the multi-port valve assembly is at its limit of travel in direction 102, passageway 74 is blocked by valve ring 116 from communicating with chamber 104, passageway 76 is blocked by valve ring 123 from communicating with chamber 106, passageway 72 is not blocked by valve ring 116 and communicates freely with chamber 104, and passageway 78 is not blocked by valve ring 123 and freely communicates with chamber 106. When the multi-port valve assembly is at its limit of travel in direction 100, passageway 72 is blocked by valve ring 116 from communicating with chamber 104, passageway 78 is blocked by valve ring 123 from communicating with chamber 106, passageway 74 is not blocked by valve ring 116 and communicates freely with chamber 104, and passageway 76 is not blocked by valve ring 123 and freely communicates with chamber 106. Such a system makes said valve assembly substantially hydraulically balanced, i.e., the valve actuation force does not vary with the case pressure or the differential pressure from chamber 104 to chamber. What hydraulic imbalance that remains is due to the cross-sectional area of valve rods 118. The exemplary embodiment of FIG. 3 explains how to eliminate the hydraulic imbalance of valve rods 118.

When the valve assembly shifts from one resting position to its other resting position, the overlap of valve ring 116 blocks both passageway 74 and passageway 72. Additionally, the overlap of valve ring 123 blocks both passageway 78 and passageway 76. Thus, passageway 74 and passageway 72 never communicate with one another through chamber 104, and also passageway 78 and 76 never communicate with one another through chamber 106. This feature increases the efficiency and accuracy of a fluid motor by eliminating internal leakage.

In the embodiment shown, an exemplary close coupled pump is mounted to a fluid motor and has an exemplary housing comprised of pump end cap 182, pump case 176, pump end cap 164, sealed by o-rings 174, and fixed together by fasteners 162. The interior of said housing is divided into a variable volume chamber 152 and a variable volume chamber 150 by a piston assembly comprised by piston half 148, piston half 146, fastener 144, actuating o-ring 156, piston ring 154, and shaft 90.

In use, a pumped fluid may enter inlet port 172 in direction 84 into inlet passageway 170. A pumped fluid may enter chamber 152 from passageway 170 through check valve 184 and then through passageway 158. Pumped fluid may enter chamber 150 from passageway 170 through check valve 186 and then through passageway 166. A pumped fluid may exit chamber 150 through passageway 168 and then through check valve 190 into outlet passageway 178. A pumped fluid may exit chamber 152 through passageway 160 and then through check valve 188 into outlet passageway 178. A pumped fluid will exit from outlet passageway 178 and through outlet port 180 in direction 84.

Absent any countermeasures, a shaft between the piston in the pump and the piston in the motor may change the displaced volume from one stroke direction to another stroke direction. This change may be disadvantageous, particularly in proportioning applications. As a countermeasure, a displacement compensator may incorporated in an embodiment to provide a substantially equivalent displacement volume. In the exemplary embodiment shown, the displacement compensator includes compensator member 218, compensator chamber 220, and compensator passageway 222. Compensator member 218 may mount to piston 98 and may slidably, sealably pierce endcap 224. The cross section of compensator 218 functionally reduces the effective cross section of piston 98 in chamber 106. When compensator member 218 moves in direction 100 during a stroke, a powering fluid enters chamber 220 from inlet passageway 78 through compensator passageway 222 to replace the displaced volume. When compensator member 218 moves in direction 102 during a stroke, compensator member 218 displaces a powering fluid in chamber 220 that exits through compensator passageway 222 into inlet passageway 78. Thus, the compensator feature reduces the stroke volume of chamber 106. The dimensions of the compensator member 218 may be any size appropriate for the pump and motor, and may be selected so that the ratio of displaced volume from pump chamber 152 to displaced volume of motor chamber 104 in stroke direction 100 will be the same as the displaced volume of pump chamber 150 to displaced volume of motor chamber 106 in stroke direction 102.

In use, a powering fluid may enter inlet port 82 and continue through inlet manifold 80 to enter passageway 74, as passageway 78 is blocked. From passageway 74, a powering fluid enters chamber 104 causing piston 98 to move in direction 102, as passageway 72 is blocked. As piston 98 moves in direction 102, chamber 106 decreases in volume and a powering fluid within is expelled through passageway 76, as passageway 78 is blocked. The powering fluid continues from passage 76 into outlet manifold 78 and then exits through outlet port 79, as passageway 72 is blocked. Continued advance of piston 98 in direction 102 will cause contact with, and then compression of spring 124. Continued advance of piston 98 in direction 102 will further compress spring 124 between piston 98 and valve ring 123 since valve ring 123 is restrained from movement in direction 102 by roller 109 of the above-described exemplary release mechanism. A powering fluid is displaced from chamber 220 by the advance of member 218. The displaced fluid exits chamber 220 by means of passageway 222 into inlet passageway 78.

The movement of the fluid motor piston assembly in direction 102 may cause a movement of the fluid pump piston assembly in the exemplary fluid pump in direction 102 due to a linkage provided by shaft 90. Movement of the fluid pump piston causes an increase in volume for pump chamber 152 and a decrease in volume for pump chamber 150. Check valve 184 allows flow from inlet port 172 through passageway 170, check valve 184 and passageway 158 into chamber 152 while check valve 188 blocks flow from outlet port 180 through passageway 178, check valve 188 and passageway 160 into chamber 152. Check valve 190 allows flow to outlet port 180 through passageway 168, check valve 190 and passageway 178 from chamber 150 while check valve 186 blocks flow into inlet port 172 through passageway 166, check valve 186 and passageway 170 from chamber 150.

Various static and dynamic seals are anticipated for various products based on the present invention. Some models may use gaskets, adhesives, o-rings or various combinations for static seals. Some models may use a close running fit, piston ring, packing, o-ring, lip seal or combinations for the dynamic seals of the pistons and rods. Other seals may be used. A variety of such seals are commercially available.

Some embodiments of the present invention may include multiple pumps operated by a single fluid motor. For example, in addition to the exemplary pump shown in FIG. 7, one or more additional pumps may be added by shaft extensions and pistons operating in stacked chambers as simply as the repetition of pump housing segments 182, 176, and 164. To add an additional pump, some modification may be required. For example, in one exemplary embodiment, segment 164 includes a hole for passage of a shaft extension. It should be noted that the chambers and pistons need not have the same cross section or displacement per stroke. Any type of pump requiring a reciprocation motion is suitable for mounting to the fluid motor of this invention. Some examples of such pumps include the piston pump shown, and also include a diaphragm pump having a diaphragm operable by the reciprocating motion. Other types of pumps may also be used.

In the exemplary embodiments shown, the check valves of the pump are shown as cartridge type. However, the check valves could be any type. The choice of check valve used will depend on the application characteristics desired for the pump, principally pressure, duty cycle, and material compatibility.

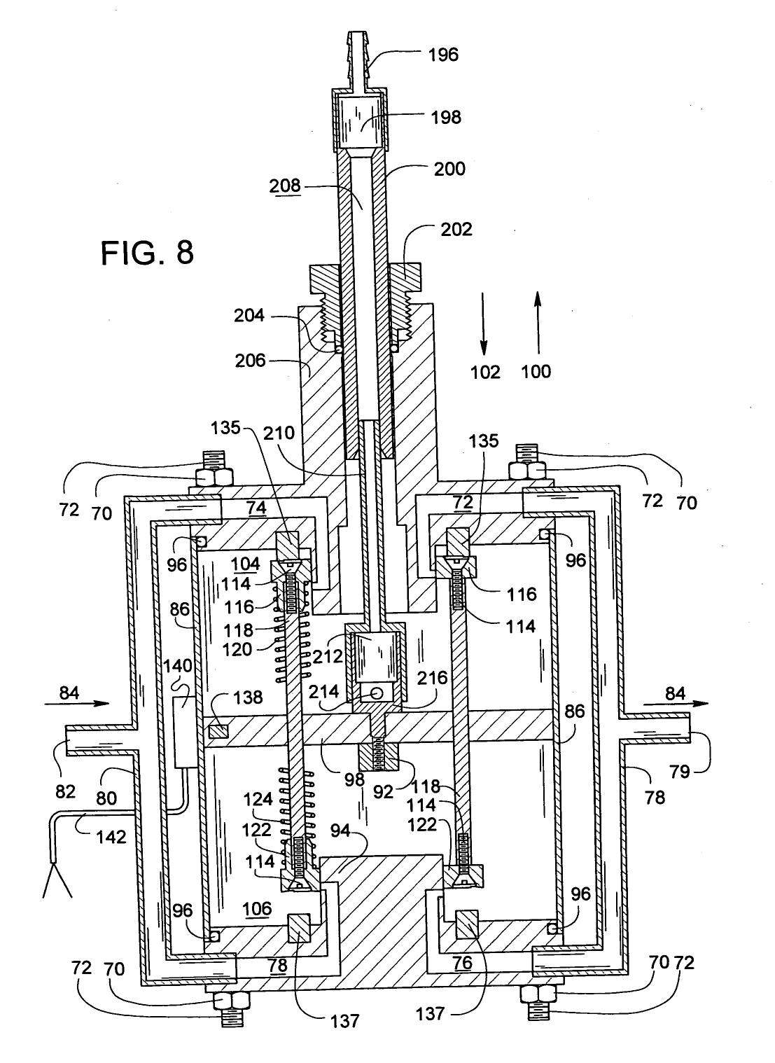

FIG. 8 shows a right view, cross section of the exemplary motor of FIG. 6 with modifications including the addition of an exemplary internal, single acting, adjustable displacement, fluid pump. An exemplary housing assembly may be comprised of a case 86 with a cylindrical interior surface. One end of the case may be closed by end cap 206 and the other end may be closed by end cap 94. The interior of the housing may be sealed by a sealing material, such as, for example, by o-rings seals 96. The exemplary housing assembly comprised of case 86, end cap 206, end cap 94 and seals 96 is held together by threaded tie rods 72 and nuts 70. However, other methods or systems may be used to hold the housing together, as would be apparent to one skilled in the art.

In use, a powering fluid enters inlet port 82 in direction 84. A powering fluid is delivered to end cap 206 and end cap 94 through inlet manifold 80. A powering fluid is collected from end cap 206 and end cap 94 through outlet manifold 78. A powering fluid leaves through outlet port 79 in direction 84. The interior of a housing assembly is divided into a variable volume chamber 104 and another variable volume chamber 106 by a piston 98.

In FIG. 8, an exemplary multi-port valve assembly is comprised of valve ring 116, valve ring 122, valve connecting rods 118, actuating spring 120, actuating spring 124, and screw fasteners 114. In the exemplary embodiment shown, valve ring 116 is connected to valve ring 122 by valve connecting rods 118 that slidably pierce piston 98. Screw fasteners 114 fix rods 118 to ring 116 and ring 122. A boss on ring 116 supports actuating spring 120. A boss on ring 122 supports actuating spring 124.

In this exemplary embodiment, a magnet 138 is attached to piston 98 to establish a magnetic field that is detectable outside case 86 by detector 140. Signal cable 142 connects detector 140 to additional signal processing device such as a flow rate meter, flow totalizing meter, or sending unit. Because the stroke, and therefore displacement per stroke, are well defined, counting the strokes may provide good correlation for a calculated flow as compared with the actual flow. Other methods of determining the flow may be used.

In FIG. 8, an exemplary adjustable displacement internal pump is comprised of inlet hose barb 196, cartridge check valve 198, pump case 200, retaining nut 202, o-ring seal 204, pump piston 210, cartridge check valve 212, pump base 216, and pump fastener 92. Other pumps having other components could be also be used. In the exemplary pump shown, pump piston 210, cartridge check valve 212, pump base 216, and pump fastener 92 comprise a pump subassembly that is fixed to piston 98 and moves with it. Additionally, inlet hose barb 196, cartridge check valve 198, pump case 200, retaining nut 202, o-ring seal 204 comprise a pump subassembly that has a static position in relationship to end cap 206. However, said pump subassembly is slidably positionable in direction 102 and direction 100 with respect to end cap 206 for the purpose of adjusting the pump displacement. Slidably positioning said pump subassembly in direction 100 decreases the pump displacement per piston stroke while slidably positioning in direction 102 increases the pump displacement per piston stroke.

In use, a powering fluid enters inlet port 82 and continues through inlet manifold 80 to enter passageway 74, as passageway 78 is blocked. From passageway 74, a powering fluid enters chamber 104 causing piston 98 to move in direction 102, as passageway 72 is blocked. As piston 98 moves in direction 102, chamber 106 decreases in volume and a powering fluid within is expelled through passageway 76, as passageway 78 is blocked. A powering fluid continues from passage 76 into outlet manifold 78 and then exits through outlet port 79, as passageway 72 is blocked. Because of the fluid pressure, piston 98 advances in direction 102. In the exemplary embodiment shown, the piston 98 will continue its advance and will compress actuating spring 124. While spring 124 undergoes compression, the multi-port valve assembly comprised of valve ring 116, valve ring 122, rods 118, and fasteners 114 may be held at its limit of travel position in direction 100 by the attractive force between magnet 135 and valve ring 116. The attractive force between magnet 137 and valve ring 122 may be comparatively weak because of the distance separating them. As spring 124 is further compressed by the advance of piston 98 in direction 102, the stored energy and force in the spring 124 increases. By design, the force of spring 124 will at some point first equal, and then exceed, the countering force of magnetic attraction between magnet 135 and valve ring 116. When this happens, the valve assembly will rapidly shift in direction 102 by the force of spring 124. When the valve assembly reaches its limit of travel position in direction 102, the attractive force between magnet 137 and valve ring 122 operate to maintain it in a position there. The shifting of the multi-port valve assembly re-directs a powering fluid flow causing piston 98 to reverse its stroke direction. The reciprocating cycle may then repeat.

As piston 98 moves in direction 102, pump piston 210 withdraws from pump chamber 208. A pumped fluid enters inlet barb 196 continues through check valve 198 into chamber 208 to compensate the displacement of pump piston 210 as long as it is slidably engaged with the bore of pump case 200 providing a seal between chamber 208 and 104. Although chamber 208 and chamber 104 communicate through pump passageway 214, check valve 212 and an interior passageway of pump piston 210, check valve 212 prevents the flow of a powering fluid in chamber 104 from entering chamber 208 by this pathway. However, when piston 98 reverses direction, pump piston 210 enters pump chamber 208 displacing the pumped fluid within and forcing it to exit through check valve 212, passageway 214 and into motor chamber 104. Such an operation allows the pump fluid and the powering fluid to mix in chamber 104. The mixed fluids may be discharged together through passageway 72, outlet manifold 78 and motor-outlet port 79. In this way, two fluids may be proportioned and mixed. The volume of pumped fluid is varied by the slidable position of pump case 200.

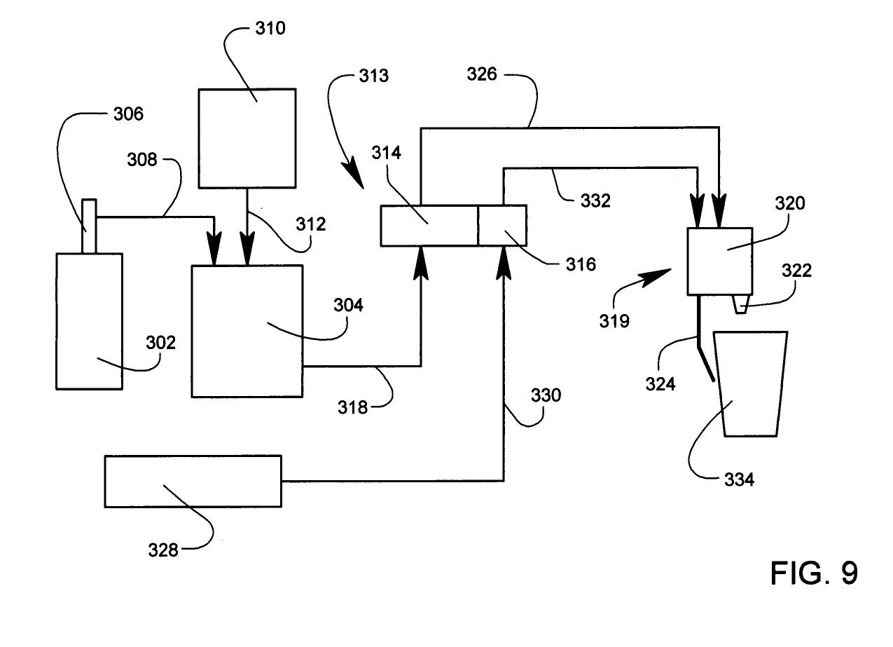

FIG. 9 is a schematic of an exemplary apparatus with its most basic elements and a single dispenser valve. This apparatus includes an example of a portion of an exemplary complete post mix dispenser. In the dispenser, a pressurized C02 gas supply 302 may communicate with a carbonator 304 through a gas regulator 306 and a C02 supply line 308. A water source 310 may supply water to carbonator 304 through water supply line 312. A carbonator 304 is typically comprised of a stainless steel tank, pump, motor, high-low level switch, relief valve and check valve. Water and C02 gas are intimately mixed under pressure and a portion of the C02 gas dissolves into the water to form a carbonated water. The tank pressure typically varies from 75 psi to 130 psi depending on the gas pressure setting and water level in the tank. However, other tank pressures may be used.

Carbonator 304 communicates with a fluid powered proportion pump 313 comprised of a fluid motor portion 314 and fluid pump portion 316 through carbonated water supply line 318. In one exemplary embodiment, the fluid powered proportioning pump 313 includes the fluid motor and the fluid pump described with reference to FIGS. 1-7. However, other proportioning pumps may be used. The fluid powered proportion pump 313 may communicate with a dispensing valve 319 comprised of valve body 320, valve nozzle 322 and actuating lever 324 through carbonated water line 326.

A syrup source 328 communicates with the fluid powered proportioning pump 313 through a syrup supply line 330. The fluid powered proportioning pump 313 communicates with the dispensing valve 319 through syrup line 332.

In this exemplary embodiment, dispensing of a drink is initiated by pressing a cup 334 against actuating lever 324. Actuating lever 324 then opens a syrup valve and carbonated water valve within valve body 320. Syrup and carbonated water under pressure flow through nozzle 322 and pour into a cup 334 in a ratio, such as 1 to 5, supplied by the fluid powered proportioning pump 313. A differential pressure from a carbonator tank (having an exemplary pressure in the range of 75 to 130 psig) to a cup 334 (0 psig) causes a flow of carbonated water from the carbonator tank of carbonator 304 to cup 334. This flow of carbonated water passes through the fluid motor 314 of the fluid powered proportion pump 313 where an amount (for example, approximately 10 psi) of the available pressure is used to power the pump 316 (which may be a reciprocating pump) of the fluid powered proportioning pump 313. The syrup is pumped from the syrup source 328 to the cup 334 by a pressure differential created by the pump 316 of, for example, approximately 20 psig. Termination of drink dispensing occurs at the removal of the cup 334 from the actuating lever 324. The actuating lever 324 then closes the syrup and carbonated water valves in the valve body 320. The flow of carbonated water stops and the pressure of carbonated water in line 326 and line 318 become the same at a pressure equal to the pressure in the carbonator tank of carbonator 304. The fluid motor 314 and fluid pump 316 of the fluid powered proportioning pump 313 cease to operate and enter a stalled condition.

It should be noted that the apparatus shown in FIG. 9 may be expanded as desired to provide additional dispensing. In one exemplary embodiment, a second fluid pump portion (not shown) may be connected to the fluid pump portion 316 or the fluid motor 314 for pumping a second syrup source (not shown). The second fluid pump may, like the fluid pump portion 316, be driven by the fluid motor 314. Alternatively, the second fluid pump may be separately driven by a second fluid motor (not shown). The second fluid pump may be connected to the dispensing valve 319 for dispensing of the second syrup simultaneously with the syrup from the syrup source 328. The ratio of syrup from each source to each other and to the carbonated water may be adjusted as desired to provide a desired mixture.

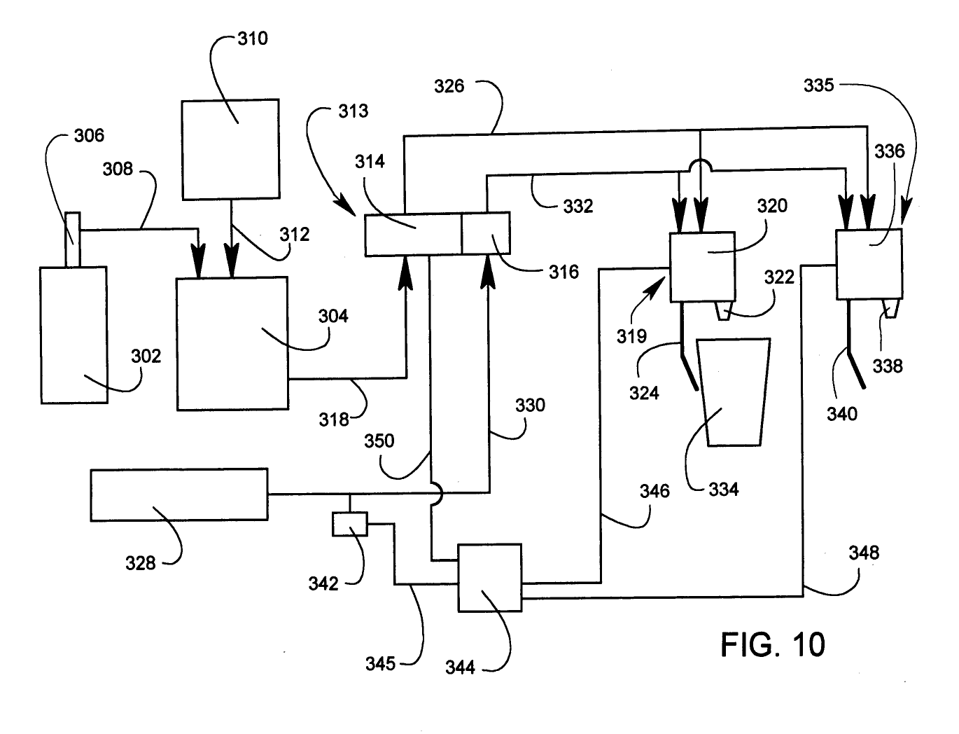

Referring to FIG. 10, the same events occur as described with reference to FIG. 9 when the cup 334 operates an exemplary dispensing valve 335 comprised of a valve body 336, a valve nozzle 338 and an actuating lever 340. The dispensing valve, such as dispensing valve 319 (described above) or the dispensing valve 335 may include two component valves, one for water and one for syrup. The water and syrup valves may be operated by a mechanical linkage with an actuating lever 324 or operated by solenoids energized by a switch controlled by actuating lever 324. However, other valves may be used so that with the present invention, it is not always necessary to use two component valves. For example, in some cases, a dispensing valve may include a component water valve without a syrup valve. In such an embodiment, when flow of carbonated water is blocked by the component water valve, the fluid powered proportioning pump 313 ceases to operate and the flow of syrup likewise ceases. The exception is where a dispensing valve is located at an elevation substantially below the syrup source elevation and siphoning occurs through check valves of a fluid powered proportioning pump 313.

FIG. 10 is a schematic of showing the apparatus of FIG. 9 with additional features including multiple dispensing valves and a control mechanism. This apparatus includes a portion of a complete post mix dispenser. The apparatus includes the pressurized C02 gas supply 302 in communication with the carbonator 304 through the gas regulator 306 and the C02 supply line 308. The water source 310 supplies water to the carbonator 304 through the water supply line 312. The carbonator 304 may be comprised of a stainless steel tank, a carbonator pump, a carbonator motor, a high-low level switch that operates a carbonator motor, a relief valve, and a check valve. Water and C02 gas may be intimately mixed under pressure and a portion of the C02 gas dissolves into the water to form a carbonated water. The carbonator tank pressure typically varies from 75 psi to 130 psi depending on the gas pressure setting and water level in the tank. However, other pressures may be used. The fluid motor 314 and the fluid pump 316 comprise the fluid powered proportioning pump 313. The carbonator 304 may communicate with the fluid motor 314 of the fluid powered proportion pump 313 through the carbonated water supply line 318. The fluid motor 314 of the fluid powered proportion pump 313 communicates with the dispensing valve 319 including the valve body 320, the valve nozzle 322, and the actuating lever 324 through the carbonated water line 326. An additional dispensing valve 335, which may include a valve body 336, a valve nozzle 338, and an actuating lever 340, may communicate with the fluid motor 314 of the fluid powered proportioning pump 313 through a parallel connection of carbonated water line 326.

The syrup source 328 may communicate with the fluid pump 316 of the fluid powered proportioning pump 313 through syrup supply line 330. The fluid pump 316 may communicate with the dispensing valve 319 including the valve body 320, the valve nozzle 322, and the actuating lever 324 through the syrup supply line 332. An additional dispensing valve 335, including the valve body 336, the valve nozzle 338, and the actuating lever 340 may communicate with the fluid pump 316 of the fluid powered proportioning pump 313 through a parallel connection of syrup supply line 332.

A vacuum switch 342 may be included to sense the pressure in the syrup supply line 330. The vacuum switch 342 may provide an electrical signal to a controller 344 via wiring 345. The proportioning pump 313, including fluid motor 314 and fluid pump 316, may provide an electrical signal to controller 344 through wiring 350. Controller 344 may control power to solenoid valves that may be housed within the dispensing valve 319, which includes the valve body 320, the valve nozzle 322, and the actuating lever 324, through a control wire 346. The controller 344 may also control power to solenoid valves that may be housed within the dispensing valve 335, which may include the valve body 336, the valve nozzle 338, and the actuating lever 340, through a control wire 348. The dispensing valves 319, 335 may provide an electrical signal through control wires 346, 348, respectively.

In one exemplary embodiment, the vacuum switch 342 may be used to sense the depletion of a syrup source. In this embodiment, the pressure in the syrup supply line 330 falls lower and lower as the fluid pump 316 displaces a volume that cannot be replaced by the fluid volume of a syrup source 328, due to a lack of syrup.

In use, dispensing of a drink is initiated by pressing the cup 334 against the actuating lever 324. The actuating lever 324 then opens the syrup and carbonated water valves within the valve body 320. Syrup and carbonated water under pressure flow through the nozzle 322 and pour into the cup 334 in a ratio, for example, 1 to 5, supplied by the fluid powered proportioning pump 313. A differential pressure from the carbonator tank (having an exemplary pressure of 75 to 130 psig) to the cup 334 (0 psig) causes a flow of carbonated water from a carbonator tank of the carbonator 304 to the cup 334. This flow of carbonated water passes through the fluid motor 314 of the fluid powered proportion pump 313 where an amount of pressure (for example, approximately 10 psi of the available pressure) is used to power the reciprocating pump 316 of the fluid powered proportioning pump 313. The syrup may be pumped from the syrup source 328 to the cup 334 by a pressure differential created by the pump 316 of approximately 20 psig, for example.

Termination of a drink dispensing may occur at the removal of the cup 334 from the actuating lever 324. The actuating lever 324 then may close the syrup and carbonated water valves in the valve body 320. The flow of carbonated water stops and the pressure of carbonated water in line 326 and line 318 become the same at a pressure equal to the pressure in a carbonator tank of carbonator 304. The fluid motor 314 and the fluid pump 316 of the fluid powered proportioning pump 313 cease to operate and enter a stalled condition. The same events occur when the cup 334 activates the dispensing valve 335, including the valve body 336, the valve nozzle 338, and the actuating lever 340.

A dispensing valve, such as dispensing valves 319, 335 may include two component valves, one for water and one for syrup. The water and syrup valves of dispensing valve 319 may be operated by a mechanical linkage with an actuating lever 324 or operated by solenoids energized by a switch controlled by actuating lever 324. However, other valves may be used so that with the present invention, it is not always necessary to use two component valves. In some cases, a dispensing valve may include a component water valve without a syrup valve. In such an embodiment, when flow of carbonated water is blocked by the component water valve, the fluid powered proportioning pump 313 ceases to operate and the flow of syrup likewise ceases. The exception is where a dispensing valve is located at an elevation substantially below the syrup source elevation and siphoning occurs through check valves of a fluid powered proportioning pump 313. The same applies to additional valves (not shown), that may be included with the apparatus.

It should be noted that the apparatus shown in FIG. 10 may be expanded as desired to provide additional dispensing. In one exemplary embodiment, a second fluid pump portion (not shown) may be connected to the fluid pump portion 316 or the fluid motor 314 for pumping a second syrup source (not shown). The second fluid pump may, like the fluid pump portion 316, be driven by the fluid motor 314. Alternatively, the second fluid pump may be separately driven by a second fluid motor (not shown). The second fluid pump may be connected in the same manner as the fluid pump portion 316 to the dispensing valves 319, 335 for dispensing of the second syrup simultaneously with the syrup from the syrup source 328. The ratio of syrup from each source to each other and to the carbonated water may be adjusted as desired to provide a desired mixture.

In some exemplary embodiments, if multiple dispensing valves are operated while connected in parallel to a fluid powered proportioning pump, there may be no assurance that each valve is dispensing the correct ratio. One exemplary embodiment of the apparatus addresses this by sending a signal to the controller 344 from the first dispensing valve operated to cause the controller 344 to prohibit the operation of the remaining dispensing valves, for example, by denying them electrical power to operate their solenoids. When the operating dispensing valve completes its dispensing, then controller 344 may re-enable all dispensing valves.

Some exemplary embodiments of a dispensing valve may have a portion control feature that, for example, may allow the filling of a specific size cup, such as, for example, 12, 16, or 32 ounce, and with or without ice. The controller 344 may accept signal information for portion control of a dispenser valve and cause the dispenser valve to operate through its solenoid. Using a stroke signal from the fluid powered proportioning pump 313 through a signal wire 350, the controller 344 can calculate the drink volume dispensed and terminate operation of the dispensing valve at the appropriate moment, thereby fulfilling the portion control function.

With an appropriately enabled controller 344, other useful information may be collected and analyzed from a stroke signal from the fluid powered proportioning pump 313 through the signal wire 350. By comparing strokes per interval of time, a flow rate may be calculated. By counting strokes, the total dispensed volume of drink may be calculated for various time periods. Such information may be useful for business purposes or to enable further postmix dispenser features. Additionally, the controller 344 may be configured to process a signal from the vacuum switch 342 to operate a “sold out” indicator or alarm function. In one exemplary embodiment, the vacuum switch 342 is configured to interrupt a circuit powering solenoids of the dispenser valves to prohibit dispensing when syrup is not available.

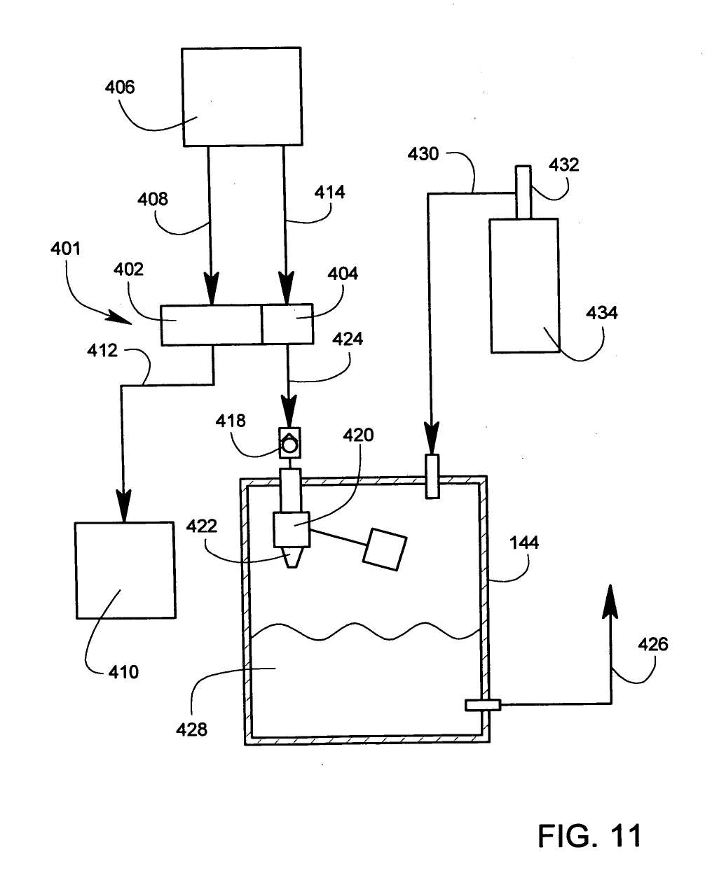

FIG. 11 is a schematic of an exemplary carbonator system for a post-mix soft drink dispenser where a fluid powered proportioning pump 401 is used as a water pressure booster. Such a system may reduce or eliminate the need for a standard carbonator pump, motor, and high-low level switch. In this exemplary embodiment, the fluid powered proportioning pump 401 includes a fluid motor 402 and fluid pump 404. The fluid powered proportioning pump 401 may also be described as a fluid powered booster pump.

In the carbonator system, a pressurized water source 406 may be a municipal water supply pipe at pressures, for example, from 40 to 80 psig. The fluid motor 402 may communicate with the pressurized water source 406 through an inlet line 408. The fluid motor 402 may also communicate with a water sink 410 through an outlet line 412. The water sink 410 may include tanks, reservoirs, and/or a drain, depending on whether the water is to be used or reused for any other purpose. The fluid motor 402 may use the pressure differential between the water source 406 and the water sink 410 as its energy source. The fluid pump 404 may communicate with the water source 406 through a pump inlet line 414. The fluid pump 404 may communicate with a carbonator comprised of a carbonator tank 416, a check valve 418, a float valve 420, and a nozzle 422 through a pump outlet line 424. A carbonated water outlet line 426 may provide a communication means for carbonated water 428 to reach its subsequent post-mix dispenser component, such as, for example, a dispensing valve, a cold plate, a fluid powered proportioning pump, or the like. The carbonator tank 416 may contain carbon dioxide gas provided through an inlet C02 gas line 430 from a C02 regulator 432 and from a C02 supply tank 434.

In use, a demand for carbonated water causes the level of carbonated water 428 in carbonator tank 416 to fall. Once it has fallen to the low level set point of the float valve 420, the float valve 420 opens. Water from water pump 404 sprays into the carbonator tank 416 through the nozzle 422. The C02 gas in carbonator tank 416 readily dissolves into the water as a result of temperature, pressure, and high surface area-contact, typical of carbonator operation. Water from the water motor 402 may flow to sink 410. In one example, if the pressure of the water source 406 is 50 psig, the pressure of the water sink 410 is 0 psig, and the ratio between the water motor 402 and the water pump 404 is 2 to 1, then pump 404 may be capable of delivering water at a pressure of approximately 150 psi. Such a pressure may be sufficient for a post-mix beverage dispensing application. When the level of carbonated water 428 rises to the high level set point of the float valve 420, the float valve 420 closes. With float valve 420 closed, water pump 404 may have no available volume to pump into and it may cease its pumping action. Since water motor 402 is directly linked to water pump 404, it may also cease its motor action. In this state, the fluid powered booster pump may have no water flowing through it, and it may remain in this stalled condition until float valve 420 re-opens. Alternatively, the float valve 420 may start and stop the fluid powered booster pump by acting on the motor inlet line 408, or the motor outlet line 412 rather than the pump outlet line 424. In one exemplary embodiment, a high-low fluid level switch operates a solenoid valve to interrupt the inlet line 408, the outlet line 412, and/or outlet line 424, and may can substitute for the float valve.

It will be apparent to those skilled in the art that various modifications and variations can be made to the structure and methodology described herein. Thus, it should be understood that the invention is not limited to the examples discussed in the specification. Rather, the present invention is intended to cover modifications and variations.

1. A fluid transfer device comprising:
a housing;
a reciprocating piston in the housing, the piston and the housing being configured to at least partially define at least two variable-volume chambers;
an inlet chamber port and an outlet chamber port associated with each of the at least two chambers;
a valve member operatively associated with each inlet chamber port and outlet chamber port to permit or prohibit fluid flow past the respective inlet chamber port and outlet chamber port, wherein said at least two chambers are in a state of fluid isolation, such that fluid in one of said at least two chambers does not enter into another of said at least two chambers;
a valve actuating means configured to shift the valve members to permit or prohibit fluid flow past the inlet and outlet chamber ports; and
a release mechanism associated with at least one valve member, the release mechanism being configured to allow the valve actuating means to shift the valve members to permit or prohibit fluid flow past the inlet or outlet chamber ports.

2. The fluid transfer device of claim 1, wherein the release mechanism is configured to be triggered by advancing the piston.

3. The fluid transfer device of claim 1, comprising a connecting member configured to link each valve member in a manner that the movement of one valve member causes a like movement in the other valve member.

4. The fluid transfer device of claim 3, wherein the connecting member is hydraulically balanced.

5. The fluid transfer device of claim 3, wherein the connecting member is configured to operate through the piston without affecting the isolation of one chamber from the other.

6. The fluid transfer device of claim 1, wherein the valve actuating means is configured to store energy and release the stored energy to shift the valve members.

7. The fluid transfer device of claim 1, wherein the valve actuating means comprises at least one spring.

8. The fluid transfer device of claim 1, wherein the valve actuating means comprises at least one magnet.

9. The fluid transfer device of claim 8, wherein the valve actuating means comprises at least two magnets oriented in a manner that the polarity of one magnet repels the other magnet.

10. The fluid transfer device of claim 1, wherein the release mechanism comprises a position biased catching member.

11. The fluid transfer device of claim 1, wherein the release mechanism is comprised of an over-the-center position biased member.

12. The fluid transfer device of claim 1, wherein the release mechanism comprises at least one magnet.

13. The fluid transfer device of claim 12, wherein the release mechanism comprises at least two magnets oriented in a manner that the polarities of said at least two magnets attract.

14. The fluid transfer device of claim 1, wherein the release mechanism comprises a spring loaded mechanical catch.

15. The fluid transfer device of claim 1, comprising a shaft fixed to the piston.

16. The fluid transfer device of claim 1, wherein the valve members are configured to substantially obstruct fluid flow from inlet chamber port to outlet chamber port while the valve members are shifted by the valve actuating means.

17. The fluid transfer device of claim 1, wherein the piston is configured to reciprocate using a gas.

18. The fluid transfer device of claim 1, wherein the piston is configured to reciprocate using a liquid.

19. The fluid transfer device of claim 1, wherein a valve member is hydraulically balanced with respect to its chamber and the pressures of the inlet and outlet.

20. The fluid transfer device of claim 1, comprising a sensor configured to detect cycling of the piston and provide a signal for informational or control purposes.

21. A fluid motor comprising:
a housing;
a reciprocating piston in the housing, the piston and the housing being configured to at least partially define first and second variable-volume chambers, the first and second chambers being separated by the piston and being maintained in fluid isolation from each other;
an inlet chamber port and an outlet chamber port associated with each of first and second chambers; and
a valve assembly comprising
a valve member operatively associated with each inlet chamber port and outlet chamber port to permit or prohibit fluid flow past the respective inlet chamber port and the outlet chamber port,
a connecting member configured to link each valve member in a manner that the movement of one valve member causes a like movement in the other valve members, and
a valve actuating mechanism configured to actuate the valve members when the piston advances to a designated set-point to reverse the movement of the piston.

22. The fluid motor of claim 21, wherein the connecting member is hydraulically balanced.

23. The fluid motor of claim 21, wherein the valve actuating mechanism comprises at least one spring.

24. The fluid motor of claim 21, wherein the valve actuating mechanism comprises at least one magnet.

25. The fluid motor of claim 24, wherein the valve actuating mechanism comprises at least two magnets oriented in a manner that the polarity of one magnet repels the other magnet.

26. The fluid motor of claim 21, comprising
a release mechanism associated with at least one valve member, the release mechanism being configured to allow the valve actuating mechanism to actuate the valve members to permit or prohibit fluid flow past the inlet or outlet chamber ports.

27. The fluid motor of claim 26, wherein the release mechanism comprises a position biased catching member.

28. The fluid motor of claim 26, wherein the release mechanism comprises a spring biased mechanical catch.

29. The fluid motor of claim 26, wherein the release mechanism comprises at least one magnet.

30. The fluid motor of claim 29, wherein the release mechanism comprises at least two magnets oriented in a manner that the polarities of said at least two magnets attract.

31. The fluid motor of claim 21, wherein the piston is configured to reciprocate using a gas.

32. The fluid motor of claim 21, wherein the piston is configured to reciprocate using a liquid.

33. The fluid motor of claim 21, wherein the valve member is hydraulically balanced with respect to its chamber and the pressures of the inlet and outlet chamber ports.

34. The fluid motor of claim 21, wherein the valve member is configured to substantially obstruct fluid flow from the inlet chamber port to the outlet chamber port while the valve members are shifted by the valve actuating mechanism.

35. The fluid motor of claim 21, comprising a sensor configured to detect cycling of the piston and provide a signal for informational or control purposes.

36. A system comprising:
the fluid motor of claim 21; and
a pump configured to be powered by the fluid motor.

37. The system of claim 36, comprising a shaft associated with the piston in a manner that the piston drives the shaft, the shaft also being associated with the pump to drive the pump.

38. The system of claim 36, wherein the pump comprises a pump piston configured to displace fluid.

39. The system of claim 36, wherein the pump comprises a diaphragm configured to displace fluid.

40. The system of claim 36, wherein the pump comprises a plurality of pumping chambers.

41. The system of claim 36, wherein the pump is at least partially disposed in the housing of the fluid motor.

42. The system of claim 36, wherein a displacement of the pump is adjustable.

43. The system of claim 36, wherein the pump is configured to pump a liquid.

44. The system of claim 36, wherein the pump is configured to pump a gas.

45. The system of claim 36, wherein the motor includes a hydraulically balanced valve.

46. The system of claim 36, wherein the fluid motor is configured to maintain a ratio of a powering fluid displacement to a pumped fluid displacement in both stroke directions.

47. The system of claim 36, comprising:
a shaft associated with the fluid motor and the pump; and
a shaft seal associated with the shaft to prohibit fluid flow along the shaft between the fluid motor and the pump.

48. The system of claim 36, comprising:
a shaft associated with the fluid motor and the pump; and
a plurality of shaft seals associated with the shaft to prohibit fluid flow along the shaft between the fluid motor and the pump.

49. The system of claim 48, comprising a chamber between at least two of the plurality of shaft seals, wherein the chamber is vented to atmosphere.

50. The system of claim 48, comprising a chamber between at least two of the plurality of shaft seals, wherein a barrier fluid occupies the chamber between seals.

51. The system of claim 48, wherein at least two of the plurality of shafts seals are spaced apart a distance greater than the distance of displacement of the shaft.

52. A method for operating a fluid motor having a first and a second fluid chamber separated by a reciprocating piston, the first and second fluid chambers each having an inlet port and an outlet port, the method comprising:
introducing fluid to the first chamber through the first chamber inlet port;
increasing the volume of the first chamber by moving the piston;
storing energy in a valve actuating means, the energy being provided by the moving piston;
releasing the stored energy in the valve actuating means with the piston;
shifting valve members with the released energy to block the first chamber inlet port and to open the second chamber inlet port;
introducing fluid to the second chamber; and
increasing the volume of the second chamber by moving the piston.

53. The method of claim 52, wherein releasing the stored energy comprises triggering a release mechanism associated with the valve members.

54. The method of claim 52, wherein storing energy in a valve actuating means comprises compressing a spring.

55. The method of claim 52, comprising
maintaining the valve members in a position with a maintaining force.

56. The method of claim 55, wherein releasing the stored energy in the valve actuating means occurs when the stored energy exceeds the maintaining force.

57. The method of claim 55, wherein the maintaining force is a magnetic force.

58. The method of claim 52, wherein each valve member is linked to the other by a connecting member that the shifting of one valve member causes a like shift in the other valve member.

59. The method of claim 52, comprising
driving an output shaft associated with the piston.

60. The method of claim 59, comprising
powering a fluid pump with the output shaft.

61. A dispensing machine comprised of at least one dispensing circuit comprising:
a fluid powered motor;
at least one fluid pump associated with the fluid powered motor such that the fluid pump is powered by the fluid powered motor;
at least one powering fluid for powering the motor;
at least one pumped fluid for pumping by the at least one fluid pump, wherein the powering fluid and the pumped fluid are separate when upstream from the fluid powered motor and the at least one fluid pump, and wherein the powering fluid is separate downstream from the fluid powered motor and the at least one fluid pump; and
at least one dispensing location disposed downstream of the fluid powered motor and the at least one fluid pump.

62. The dispenser of claim 61, wherein the at least one dispensing location includes at least two dispensing locations disposed in a parallel relationship downstream of the fluid powered motor and the at least one fluid pump.

63. The dispenser of claim 61, wherein the at least one fluid pump is configured to selectively pump the at least one pumped fluid to the dispensing location.

64. The dispenser of claim 61, wherein the dispensing location comprises a dispensing valve configured to initiate and terminate the operation of the fluid powered motor and the at least one fluid pump when the dispensing valve enabled and disabled, respectively.

65. The dispenser of claim 64, comprising a switch configured to monitor the availability of the at least one pumped fluid and configured to disable the operation of the dispensing valve when a quantity of the at least one pumped fluid falls below a preset level.

66. The dispenser of claim 61, comprising:
a vacuum switch configured to monitor availability of a pumped fluid; and
a controller means in communication with a vacuum switch,
wherein the at least one dispensing location includes a dispensing valve, and wherein the controller means is configured to disable operation of the dispensing valve when availability of the at least one pumped fluid fails below a preset level.

67. The dispenser of claim 61, comprising:
a plurality of dispenser valves disposed at the dispensing location, the plurality of dispenser valves being in fluid communication with the at least one fluid pump; and
a controller configured to monitor at least one of the plurality of dispenser valves and configured to disable operation of at least one other of the plurality of dispenser valves in response to operation of the monitored dispenser valve.

68. The dispenser of claim 61, comprising:
at least one dispenser valve disposed at the dispensing location, the at least one dispenser valve being in fluid communication with the at least one fluid pump; and
a controller configured to receive a stroke signal from at least one of the fluid powered motor and the at least one fluid pump, the controller being configured to enable and disable the at least one dispensing valve to control a quantity of fluid dispensed based at least in part on the stroke signal.

69. The dispenser of claim 61, comprising:
at least one dispenser valve disposed at the dispensing location, the at least one dispenser valve being in fluid communication with the at least one fluid pump; and
a controller configured to receive a portion signal and to enable or disable the at least one dispensing valve to control the quantity of fluid dispensed based at least in part on the portion signal.

70. The dispenser of claim 61, comprising a controller configured to process a stroke signal from at least one of the fluid powered motor and the at least one fluid pump, the controller being configured to determine process characteristics including at least one of:
flow rate of at least one of the powering fluid and the pumped fluid, and
volume of flow per a set time period of at least one of the powering fluid and a pumped fluid.

71. A post mix dispensing machine comprised of a least one dispensing circuit comprising:
a fluid powered motor;
at least one fluid pump associated with the fluid powered motor such that the fluid pump is powered by the fluid powered motor;
at least one powering fluid for powering the motor;
at least one pumped fluid for pumping by the at least one fluid pump, wherein the powering fluid and the pumped fluid are separate when upstream from the fluid powered motor and the at least one fluid pump, and wherein the powering fluid and the at least one pumped fluid is mixed downstream from the fluid powered motor and the at least one fluid pump; and
at least one dispensing location disposed downstream of the fluid powered motor and the at least one fluid pump.

72. The dispenser of claim 71, wherein the at least one dispensing location includes at least two dispensing locations disposed in a parallel relationship downstream of the fluid powered motor and the at least one fluid pump.

73. The dispenser of claim 71, wherein the at least one fluid pump is configured to selectively pump the at least one pumped fluid to the dispensing location.

74. The dispenser of claim 71, wherein the dispensing location comprises a dispensing valve configured to initiate and terminate the operation of the fluid powered motor and the at least one fluid pump when the dispensing valve enabled and disabled, respectively.

75. The dispenser of claim 74, comprising a switch configured to monitor the availability of the at least one pumped fluid and configured to disable the operation of the dispensing valve when a quantity of the at least one pumped fluid falls below a preset level.

76. The dispenser of claim 71, comprising:
a vacuum switch configured to monitor availability of a pumped fluid; and
a controller means in communication with a vacuum switch,
wherein the at least one dispensing location includes a dispensing valve, and wherein the controller means is configured to disable operation of the dispensing valve when availability of the at least one pumped fluid fails below a preset level.

77. The dispenser of claim 71, comprising:
a plurality of dispenser valves disposed at the dispensing location, the plurality of dispenser valves being in fluid communication with the at least one fluid pump; and
a controller configured to monitor at least one of the plurality of dispenser valves and configured to disable operation of at least one other of the plurality of dispenser valves in response to operation of the monitored dispenser valve.

78. The dispenser of claim 71, comprising:
at least one dispenser valve disposed at the dispensing location, the at least one dispenser valve being in fluid communication with the at least one fluid pump; and
a controller configured to receive a stroke signal from at least one of the fluid powered motor and the at least one fluid pump, the controller being configured to enable and disable the at least one dispensing valve to control a quantity of fluid dispensed based at least in part on the stroke signal.

79. The dispenser of claim 71, comprising:
at least one dispenser valve disposed at the dispensing location, the at least one dispenser valve being in fluid communication with the at least one fluid pump; and
a controller configured to receive a portion signal and to enable or disable the at least one dispensing valve to control the quantity of fluid dispensed based at least in part on the portion signal.

80. The dispenser of claim 71, comprising a controller configured to process a stroke signal from at least one of the fluid powered motor and the at least one fluid pump, the controller being configured to determine process characteristics including at least one of:
flow rate of at least one of the powering fluid and the pumped fluid, and
volume of flow per a set time period of at least one of the powering fluid and a pumped fluid.

81. The dispenser of claim 71, wherein the powering fluid is carbonated water and at least one pumped fluid is a syrup.

82. A post-mix dispenser comprising:
a fluid powered booster pump; and
a carbonator tank, wherein the fluid powered booster pump is configured to supply water to the carbonator tank.

83. The post-mix dispenser of claim 82, comprising a float valve means associated with the fluid powered booster pump in a manner to start and stop the fluid powered booster pump.
